SEQUENCE LISTING (15 sequences)

SEQ ID NO: 1 - DNA, 27 bp, Artificial Sequence
  GAG1 FAM-BHQ1
ctggcctgttagaaacatcagaaggct

SEQ ID NO: 2 - DNA, 22 bp, Artificial Sequence
  GAG1 FW
gagctagaacgattcgcagtta

SEQ ID NO: 3 - DNA, 22 bp, Artificial Sequence
  GAG1 RV
ctgtctgaagggatggttgtag

SEQ ID NO: 4 - DNA, 25 bp, Artificial Sequence
  POL1 FAM-BHQ1
tggtcagtgctggaatcaggaaagt

SEQ ID NO: 5 - DNA, 22 bp, Artificial Sequence
  POL1 FW
gtaccagcacacaaaggaattg

SEQ ID NO: 6 - DNA, 22 bp, Artificial Sequence
  POL1 RV
atgttcttcttgggccttatct

SEQ ID NO: 7 - DNA, 26 bp, Artificial Sequence
  Rev1 FAM-BHQ1
tatcaaagcaacccacctcccaatcc

SEQ ID NO: 8 - DNA, 21 bp, Artificial Sequence
  Rev1 FW
caaggcagtcagactcatcaa

SEQ ID NO: 9 - DNA, 22 bp, Artificial Sequence
  Rev1 RV
tctctctccaccttcttcttct

SEQ ID NO: 10 - DNA, 24 bp, Artificial Sequence
  VSVg1 FAM-BHQ1
atttccgctggtatggaccgaagt

SEQ ID NO: 11 - DNA, 22 bp, Artificial Sequence
  VSVg1 FW
cccaagagtcacaaggctattc

SEQ ID NO: 12 - DNA, 22 bp, Artificial Sequence
  VSVg1 RV
gagtgaaggatcggatggaatg

SEQ ID NO: 13 - DNA, 20 bp, Artificial Sequence
  LTR-P FAM-BHQ1
cagtggcgcccgaacaggga

SEQ ID NO: 14 - DNA, 20 bp, Artificial Sequence
  LTR MH531 FW
tgtgtgcccgtctgttgtgt

SEQ ID NO: 15 - DNA, 20 bp, Artificial Sequence
  LTR MH532 RV
gagtcctgcgtcgagagagc

(19) United States
(12) Patent Application Publication
(10) Pub. No.: US 2025/0122528 A1
(43) Pub. Date: Apr. 17, 2025

(54) LENTIVIRAL VECTOR

(71) Applicant: TOUCHLIGHT IP LIMITED, Hampton (GB)

(72) Inventors: Sarah MOORE, Hampton (GB); Thomas ADIE, Hampton (GB); Maria BARREIRA GONZALEZ, Hampton (GB)

(21) Appl. No.: 18/567,499

(22) PCT Filed: Jun. 8, 2022
(86) PCT No.: PCT/GB2022/051437

§ 371 Date: Dec. 6, 2023

(30) Foreign Application Priority Data
Jun. 8, 2021 (GB) 2108176.5
May 14, 2022 (GB) 2207077.5

(51) Int. Cl. C12N 15/86

(52) U.S. Cl. CPC C12N 15/86; C12N 2740/15043; C12N 2740/15052; C12N 2740/15062; C12N 2760/20222

(57) ABSTRACT

The present invention relates to a novel closed linear DNA vector, which is suitable for use in the production of lentiviral particles. Notably, the present invention relates to a new configuration of the vector including the transgene (often termed the “payload” vector), which enables a greater yield of infectious lentiviral particles, notably a greater yield of lentiviral particles carrying a trans-gene, to be prepared when compared to closed linear DNA vectors lacking this configuration. Further, the inventors have developed improvements in lentiviral production with closed linear DNA, through optimisation of vector input quantities and construct ratios. The invention furthermore relates to a method of generating infectious lentiviral particles using the construct, optionally in conjunction with improved production vectors and/or optimised methodology.

### FIELD OF THE INVENTION

The present invention relates to a novel closed linear DNA vector, which is suitable for use in the production of lentiviral particles. Notably, the present invention relates to a new configuration of the vector including the transgene (often termed the “payload” vector), which enables a greater yield of infectious lentiviral particles, notably a greater yield of lentiviral particles carrying a transgene, to be prepared when compared to closed linear DNA vectors lacking this configuration. Further, the inventors have developed improvements in lentiviral production with closed linear DNA, through optimisation of vector input quantities and construct ratios. The invention furthermore relates to a method of generating infectious lentiviral particles using the construct, optionally in conjunction with improved production vectors and/or the optimised methodology as described herein.

### BACKGROUND TO THE INVENTION

Viral vectors provide an efficient means for the modification of eukaryotic cells, and their use is now widespread in both academic laboratories and industry settings for both research and clinical gene therapy applications. The spectrum of viral vectors is very broad, and ranges from DNA viruses such as adenoviruses, to RNA viruses such as retroviruses. Lentiviruses, a genus of the Retroviridae family of viruses, are characterised by a positive-sense, single-stranded RNA genome that encodes the gag, pol and env protein-coding genes, along with the regulatory genes tat and rev. The infectious lentiviral virion enters a host cell through direct fusion with the host cell membrane or receptor-mediated endocytosis, and upon entry a lentiviral core is released and reverse transcription of the lentiviral genome takes place. The resulting double-stranded proviral DNA is subsequently integrated into the infected host cell's genome, where it relies on host machinery to initiate and complete transcription and translation of viral proteins necessary to assemble infectious particles.

Based on this framework and taking advantage of the lentivirus's highly efficient integrative capacity, lentiviral particles (LVPs) have been developed as efficient vehicles for gene transfer in mammalian cells. The vast majority of lentiviral particles are derived from the most extensively studied lentivirus, HIV-1, but other lentiviruses have also been developed as gene transfer vehicles, such as HIV-2 and simian immunodeficiency virus, and non-primate lentiviruses including feline immunodeficiency virus, bovine immunodeficiency virus and caprine arthritis-encephalitis virus.

Several generations of replication-defective lentiviral particle systems have been developed to overcome safety concerns regarding the pathogenicity of HIV-1 in humans. In principle, this has been achieved by (1) generating “minimal lentiviral genomes” through elimination of dispensable lentiviral virulence/accessory genes; (2) separating lentiviral genes/sequences essential for lentivirus generation into appropriate constructs/cassettes to minimise the possibility of generating replication-competent lentivirus. The most recently developed third-generation lentiviral (LV) systems are composed of four separate vectors: two packaging vectors encoding rev and gag-pol, where (i) rev encodes protein expression for nuclear export of the viral genome and (ii) gag and polencode for viral capsid structural proteins and the enzymes reverse transcriptase, integrase, and protease, respectively; (iii) an envelope vector encoding env, responsible for the expression of envelope glycoproteins which mediate cell entry; and (iv) a transfer vector encoding a transgene driven by a heterologous strong promoter. A third generation quadruple transfection production system is described, for example, in Dull et al, Journal Of Virology, 72 (1998), incorporated herein by reference.

A further advancement was the development of self-inactivating (SIN) constructs wherein the transfer vector contains a SIN lentiviral long terminal repeat (LTR) configuration where the homologous promoter/enhancer sequences in the U3 region of the 3′ LTR are deleted, reducing the risk of unwanted activation of genes neighbouring the lentiviral particle insertion site and decreasing the risk of lentiviral particle mobilisation.

The improved safety profile of SIN lentiviral particles, paired with their capacity to stably transduce both dividing and non-dividing cells, has facilitated the exponential growth of the use of lentiviral particles as gene therapy vectors in clinical research. However, clinical trials require large quantities of high-titre, infectious lentiviral particles, demanding highly efficient, cost-effective and scalable production methods.

Lentiviral particle synthesis can be sub-divided into two major categories, stable lentiviral particle production and transient lentiviral particle production. The former method involves the transfection of a single transgene-encoding transfer vector into a stable lentiviral particle producer cell line bearing helper functions necessary to produce functional lentiviral particles. However, difficulties in developing stable producer cell lines capable of high-titre lentiviral particle production has meant that transient lentiviral particle production has been favoured. Current methods of transient lentiviral particle production are based on the co-transfection of a permissive packaging cell line with multiple DNA vectors encoding lentiviral elements (rev, gag-pol, env) and the transfer vector. Typically, the DNA vectors are in the form of plasmid DNA (pDNA), and the preferred packaging cell is the human embryonic kidney 293 cell line (HEK293), or its derivatives (e.g. HEK293T).

The manufacture of the large quantities of high-quality DNA required for transient lentiviral particle production represents a major bottleneck in the manufacturing process, and a significant hurdle in the widespread clinical use of lentiviral particles for gene therapy. Further, there are several drawbacks associated with bio-production of lentiviral particles on a pDNA platform. GMP pDNA manufacture is costly, complex and the DNA product may ultimately be contaminated with bacterial propagation elements that are unnecessary for virus production in mammalian cells. Furthermore, eukaryotic expression cassettes may occasionally contain gene sequences that produce toxic or problematic effects in bacteria, which limits their amplification. For example, some therapeutically relevant genes are difficult to propagate in bacteria due to sequence toxicity or complexity (Feldman et al. (2014) The Nav channel bench series: plasmid preparation. Methods X., 1:6-11; McMahon et al. (2015) NIH Public Access, 27:320-31).

Synthetic in vitro amplification as described in WO2010/086626, WO2012/017210, WO2016/132129 and WO2018/033730 (and incorporated herein by reference in its entirety) is capable of producing GMP closed linear DNA vectors to multi-gram scale in 2 weeks. The resulting closed linear DNA molecules are minimal, containing only the user-defined sequences of interest, with no antibiotic resistance gene or origin of replication. Further, the use of an enzymatic DNA amplification platform to produce closed DNA vectors for lentiviral particle production could enable lentiviral particle packaging of complex DNA sequences that have previously been incompatible with bacterial propagation systems. Therefore, with their favourable safety profile, and amenability to large-scale manufacture, closed linear DNA vectors present a promising alternative to pDNA for use in lentiviral particle production.

Karda et al. (2019) have demonstrated that closed linear DNA vectors can be used to produce lentiviral particles in a second-generation lentiviral particle platform with comparable transgene expression to pDNA-derived lentiviral particles in vitro, and that titre-matched vectors have similar transgene expression in vivo. However, the infectious titre of lentiviral particles produced using closed linear DNA vectors was observed to be lower than pDNA-derived LVs. Second generation lentiviral production involves the use of a single packaging plasmid encoding the Gag, Pol, Rev, and Tat genes, an envelope plasmid encoding VSVg, and a transfer vector where transgene expression from the 5′ wild type LTR is Tat-dependent. When transferring the technology to a third generation lentiviral particle platform, wherein modified LTRs are used (both 5′ and 3′) and the dependence on Tat is removed, the applicants found that the infectious yield decreased further. Indeed, although there appeared to be both sufficiently abundant transfected DNA (FIG. 1B) and viral genome RNA in transfected cells for the generation of infectious particles (FIG. 1), this did not result in a sufficient yield of infectious lentiviral particles carrying a transgene (FIG. 2). Thus, the yield of infectious particles was not comparable to yields achieved with other DNA vector formats. To address this, experiments were initially conducted to reduce the amount of vector DNA used in the transfection.

This did have the effect of increasing total particle titre (FIG. 2B). Subsequently, studies were performed to optimise construct ratios using the decreased DNA input. These studies further increased total particle titre but did not rescue the decreased yield of infectious particles (FIG. 3).

Thus, without being bound by theory, the effect is postulated by the inventors to relate to the nature of the closed linear DNA itself. The inventors have thus developed a novel closed linear DNA vector that can be used for lentiviral particle production, yielding infectious titres considerably higher than ‘standard’ closed linear DNA vectors described in Karda et al. (2019), and resulting in infectious titres comparable to pDNA-derived infectious titres.

### SUMMARY OF THE INVENTION

The present invention relates to a novel closed linear DNA vector, which is suitable for use in the production of lentiviral particles. The novel vector has a configuration which allows for a greater yield of infectious lentiviral particles to be prepared compared to closed linear DNA vectors lacking this configuration. The invention further relates to a method of generating infectious lentiviral particles using the construct as described herein.

The present invention provides a novel lentiviral transfer vector in a closed linear DNA format. Such contains the transgene of interest to include within the lentiviral particles as an RNA molecule.

The present invention provides:
a closed linear DNA vector suitable for use as a lentiviral transfer vector, said closed linear DNA vector comprises sequences in the following order 5′ to 3′:
(a) a hybrid 5′ long terminal repeat (LTR) sequence;
(b) a promoter operably linked to a transgene;
(c) a 3′ self-inactivating (SIN) LTR sequence;
(d) a poly(A) signal sequence; and
(e) a spacer sequence.

Additional sequences may be included within the closed linear DNA vector, for example additional spacer sequences.

In the vector, the promoter and transgene, together with any additional sequences, are effectively flanked by the 5′ and 3′ LTR sequences.

Thus, the present invention provides:
a closed linear DNA vector suitable for use as a lentiviral transfer vector, said closed linear DNA vector comprising:
(a) a promoter operably linked to a transgene; and
(b) a sequence encoding a hybrid 5′ long terminal repeat (LTR) and a 3′ self-inactivating (SIN) LTR flanking the promoter and transgene; and
(c) a sequence encoding a poly(A) signal located 3′ to the 3′ SIN LTR; and
(d) a spacer sequence located 3′ to the sequence encoding a poly(A) signal.

Therefore, the novel configuration according to any description of the invention includes sequences in the lentiviral transfer vector that are 3′ to the 3′ SIN LTR sequence in order to improve the inclusion of the transgene into the lentiviral particle.

Further, the spacer sequence of the closed linear DNA vector according to any description of the invention may be a nucleotide sequence of any appropriate length. Spacer sequences are understood to be generally a sequence of non-coding DNA that may or may not have a specific sequence. The spacer sequence may be at least 100, at least 200, at least 300, at least 400, at least 500, at least 600, at least 700, at least 800, at least 900, at least 1000, at least 1100, at least 1200, at least 1300, at least 1400 or at least 1500 nucleotides in length. Optionally the spacer sequence may be any range of nucleotides in length as disclosed here. It may be preferred that the spacer is at least 250, at least 500 or most preferably at least 1000 nucleotides in length (1kb).

The LTR sequences included within the closed linear DNA vector as described here in any aspect are both modified from wild-type LTR sequences. The 5′ LTR is a hybrid sequence, wherein said 5′ LTR is modified, optionally by replacing all or part of the U3 region with a heterologous promoter. The 3′ LTR is also modified, such that the LVPs produced are self-inactivating (SIN). This usually involves the deletion of all or part of the U3 region of the 3′ LTR. Such modifications to the LTRs are made to improve the safety of LVPs by (1) eliminating the requirement for the viral gene tat for transcription of the viral genome, thereby reducing the potential for the emergence of replication competent retroviruses (RCR) through recombination events during production, and (2) to eliminate the risk of insertional mutagenesis through LTR enhancer activity. The modifications to the LTR sequences represent the difference between second generation (wild type LTR) and third generation (modified LTR) lentiviral transfer vectors.

Further, the sequence encoding the poly(A) signal (or polyA signal sequence) in the closed linear DNA vector as described in any aspect herein may be for a strong poly(A) signal. The strong poly(A) signal is one which provides an efficient termination of transcription. Those skilled in the art will be aware of appropriate poly(A) signals, such as a Simian Virus 40 (SV40) Late poly(A) sequence, bovine growth hormone poly(A) (bGHpA), rabbit P-globin (rbGlob), or a sequence having at least 90% homology thereto.

Further, the sequence encoding a poly(A) signal (or polyA signal sequence) in the closed linear DNA vector as described in any aspect herein may include additional helper sequences, optionally wherein said helper sequences are one or more upstream sequence elements (USE). The USE may act to enhance the efficiency of the poly(A) signal.

Further, the sequence encoding the 3′ SIN LTR (or 3′SIN LTR sequence) in the closed linear DNA vector as described in any aspect herein contains deletions compared to a wild-type LTR. Optionally, said deletion is in the U3 region of the 3′ LTR, either in whole or part. Optionally, the 3′ SIN LTR contains a 133 nucleotide U3 deletion compared to a wild-type 3′ LTR, at nucleotide position −149 to −9 with respect to the transcription start site. The 3′ LTR sequence can be further modified by deletion or insertion as required. In a preferred embodiment, the modified 5′ and 3′ LTRs are derived from HIV-1. If alternative LTRs are employed, similar deletions and insertions can be made by those skilled in the art to reach the same effect.

Further, the closed linear DNA vector as described in any aspect herein may include other sequences for other elements which may be beneficial in the production of infectious lentiviral particles. These other elements are described herein, and include, but are not limited to any one or more of: WPRE, Psi, RRE, cPPT, GAG, POL, ENV, REV or any other packaging element.

Furthermore, the closed linear DNA vector may additionally comprise one or more further spacer sequences. An additional spacer sequence is preferably a 5′ spacer sequence, for example, it is positioned 5′ to the sequence for the 5′ LTR. The further spacer sequence may be a nucleotide sequence of any appropriate length. Spacer sequences are understood to be generally a sequence of non-coding DNA that may or may not have a specific sequence. The spacer sequence may be at least 100, at least 200, at least 300, at least 400, at least 500, at least 600, at least 700, at least 800, at least 900, at least 1000, at least 1100, at least 1200, at least 1300, at least 1400 or at least 1500 nucleotides in length. Optionally the spacer sequence may be any range of nucleotides in length as disclosed here. It may be preferred that the spacer is at least 250, at least 500 or most preferably at least 1000 nucleotides in length (1kb). Where the closed linear DNA vector comprises more than one spacer sequence, the spacer sequences may be the same or different nucleotide sequence and may be the same or different lengths.

The closed linear DNA transfer vector as described in any aspect herein provides the template for the RNA lentiviral “genome” which is inserted into the particle during production. Thus, some of the sequences in the closed linear DNA vector (such as the transgene) are the template for the relevant RNA sequences which are packaged into the particle. The DNA vector thus provides the relevant code for the RNA sequence. References to “coding for” in this regard will be understood to mean that the sequences of the closed linear DNA vector code for single stranded RNA (ssRNA). Thus, the closed linear DNA vector includes all the instructions to produce the correct single-stranded RNA for inclusion into the lentiviral particle. Thus, the closed linear DNA vector includes sequences for the transgene and an operably linked promoter, the 5′ LTR and 3′ SIN LTR in the RNA.

Effectively, the 5′LTR and 3′SIN LTR form the “flanking ends” of the lentiviral RNA, and thus sequences between these two elements in the closed linear DNA vector will form the lentiviral ssRNA for packaging. In conventional LV vectors, the ssRNA is then reverse-transcribed to give a double-stranded DNA (dsDNA) product, which then enters the nucleus of the transfected cell. Thus, the closed linear DNA vector includes the same sequence for the promoter and the transgene in the reverse-transcribed DNA in this instance. However, newer variants permit the ssRNA of the LV particle to be used as mRNA in the transfected cell.

It will be understood by those skilled in the art that the other sequences included in the closed linear transfer vector that are not located between (or flanked by) the 5′LTR and 3′SIN LTR are not included in the ssRNA inserted into the LV particle. Thus, although the polyA signal sequence and spacer sequences from the closed linear transfer vector are transcribed in the producer cell, the RNA is then efficiently processed to form the final RNA molecule for packaging, flanked by the LTRs.

Thus, in terms of the closed linear transfer vector, this may be described as containing sequences such as one or more of: the 5′LTR, the 3′ SIN LTR, the transgene (also called the payload), the promoter, the polyA signal sequence; the spacer sequence and any additional sequence, or it may alternatively be described as coding for such sequences, since the manufacture involves the transcription of the sequences of the closed linear DNA vector into an RNA molecule.

The closed linear DNA vector described herein is the lentiviral transfer vector, which includes the payload sequence, or transgene. However, the inventors have determined that these modifications may also apply to the production vectors, these vectors being required for the production of the lentiviral particles.

Thus, the modifications described above may also apply to one or more production vectors if they are formatted as closed linear DNA vectors. Therefore, a closed linear DNA production vector may include one or more spacer sequences. Said spacer sequence(s) may be 3′ to the gene/termination sequence/expression cassette and/or 5′ to said sequences.

Thus, the present invention includes: A closed linear DNA vector suitable for use as a lentiviral production vector, said closed linear DNA vector comprising:
(a) at least one expression cassette including one or more of:
(i) a lentiviral group specific antigen (GAG) gene;
(ii) a lentiviral polymerase (POL) gene;
(iii) an envelope gene (ENV); and/or
(iv) a lentiviral regulatory gene (REV), and
(b) a spacer sequence located 3′ to the expression cassette.

Preferably the envelope gene (ENV) is a Vesicular Stomatitis Virus Glycoprotein (VSV-G) gene.

As used herein in an expression cassette can be as minimal as a promoter operably linked to a transgene, or it may include additional sequences such as termination sequences. As used herein, termination sequences may include a polyA signal sequence or use the 3′ SIN LTR.

A spacer sequence may thus be 3′ and/or 5′ to the expression cassette.

Further, the spacer sequence of the closed linear DNA vector may be a nucleotide sequence of any appropriate length. Spacer sequences are understood to be generally a sequence of non-coding DNA that may or may not have a specific sequence. The spacer sequence may be at least 100, at least 200, at least 300, at least 400, at least 500, at least 600, at least 700, at least 800, at least 900, at least 1000, at least 1100, at least 1200, at least 1300, at least 1400 or at least 1500 nucleotides in length. Optionally the spacer sequence may be any range of nucleotides in length as disclosed here. It may be preferred that the spacer is at least 250, at least 500 or most preferably at least 1000 nucleotides in length (1kb). Where the closed linear DNA vector comprises more than one spacer sequence, the spacer sequences may be the same or different nucleotide sequence and may be the same or different lengths.

It may be preferred that the lentiviral production vector comprising an expression cassette encoding GAG/POL or REV includes a spacer sequence, 3′ to the expression cassette. It may be preferred that the closed linear DNA vector for use as a lentiviral production vector comprising an expression cassette encoding GAG/POL or REV includes a spacer sequence, 3′ to the expression cassette.

Optionally, the present invention relates to a set of closed linear DNA vectors suitable for use in the productions of lentiviral particles which comprises at least one lentiviral transfer vector as described herein and at least one lentiviral production vector as described herein. Optionally, the set of closed linear DNA vectors includes a lentiviral transfer vector as described herein together with at least three lentiviral production vectors as described herein. The three lentiviral production vectors may separately encode GAG/POL, ENV and REV. Any or all of the vectors may include a 3′ spacer sequence as defined herein.

One or more of the closed linear DNA vectors of the present invention may be used to improve production of lentiviral particles. At least the closed linear DNA transfer vector may be used to improve infectious titre.

There is thus provided a method of improving infectious titre of lentiviral particles when the transfer (payload) vector is a closed linear DNA vector, comprising introducing to a packaging cell or producer cell the novel closed linear DNA vector as described in any aspect herein (also referred to as a ‘closed linear transfer vector’).

In addition to the novel closed linear DNA transfer vector as described herein, the method of producing lentiviral particles may further comprise introducing one or more production vectors to a packaging cell. The one or more production vectors encode for viral elements required for the manufacture of lentiviral particles. The one or more production vectors encode for one or more of the following:
(a) a lentiviral group specific antigen (GAG) gene; and/or
(b) a lentiviral polymerase (POL) gene; and/or
(c) an envelope gene (ENV); and/or
(d) a lentiviral regulatory gene (REV).

Preferably the envelope gene (ENV) is a Vesicular Stomatitis Virus Glycoprotein (VSV-G) gene. The VSV-G envelope protein enables broad tropism over a range of species and cell types.

The GAG gene and POL gene may be encoded or included on a single production vector.

Further, any one or more of the genes listed as (a) to (d) above may not be required on a separate production vector or be already present in a producer cell, as the gene may be supplied on the closed linear DNA vector instead.

Further, the production vectors may be any suitable format, such as closed linear DNA vectors or circular DNA vectors, or a mixture thereof.

If the production vectors are closed linear DNA, it is preferable that they include at least one spacer sequence. A spacer sequence may preferably be included in the vector 3′ to the gene or expression cassette. The spacer sequence of the closed linear DNA vector may be a nucleotide sequence of any appropriate length. Spacer sequences are understood to be generally a sequence of non-coding DNA that may or may not have a specific sequence. The spacer sequence may be at least 100, at least 200, at least 300, at least 400, at least 500, at least 600, at least 700, at least 800, at least 900, at least 1000, at least 1100, at least 1200, at least 1300, at least 1400 or at least 1500 nucleotides in length. Optionally the spacer sequence may be any range of nucleotides in length as disclosed here. It may be preferred that the spacer is at least 250, at least 500 or most preferably at least 1000 nucleotides in length (1kb). Alternatively or additionally, the spacer sequence may be present 5′ to the gene or expression cassette.

Further, the packaging cell is a permissive cell. Optionally, the packaging cell is a HEK293 cell or a variant or derivative thereof.

Further, wherein a producer cell is used, the producer cell is a stable cell line expressing the accessory packaging functions required for lentiviral particle production.

Further, the closed linear transfer vector and/or production vectors may be introduced to the packaging cell or producer cell via any suitable means, such as transfection, optionally chemical transfection. Wherein the closed linear transfer vector and/or production vectors are introduced to the packaging cell or producer cell via chemical transfection, the transfection agent may be selected from any one of calcium phosphate (CaPO4), polyethylenimine (PEI), or lipofectamine.

Further, there is provided a method for producing and harvesting lentiviral particles from packaging or producer cells prepared according to the methods of the present invention. This method comprises:
(a) inducing production of the lentiviral particles in packaging cells transfected with at least one closed linear DNA vector adapted for the production of lentiviral particles; and/or
(b) culturing the transfected packaging or producer cells; and
(c) harvesting/isolating the produced recombinant lentiviral particles from the culture medium.

Said method may also comprise the use of one or more closed linear production vectors as described anywhere herein. Thus, said method may further comprise the use of one or more closed linear DNA production vectors, said vectors comprising:
(a) at least one expression cassette including one or more of:
(i) a lentiviral group specific antigen (GAG) gene;
(ii) a lentiviral polymerase (POL) gene;
(iii) an envelope gene (ENV); and/or
(iv) a lentiviral regulatory gene (REV), and
(b) a spacer sequence located 3′ to the expression cassette.

Preferably the envelope gene (ENV) is a Vesicular Stomatitis Virus Glycoprotein (VSV-G) gene.

Said expression cassette is as described previously.

Further, the method of producing the lentiviral particle or the method of improving the infectious lentiviral titre using the closed linear DNA transfer vector may be optimised by altering the total amount of DNA used for the transfection of the cell, notably by reducing the total amount of DNA used for transfection of the cell. This reduction is achieved when compared to other DNA vector types such as plasmids. Preferably, the total DNA transfected is less than 1 μg/ml, less than 0.9 μg/ml, less than 0.8 μg/ml or less than 0.75 μg/ml. Optimally, the total amount of DNA transfected is 0.7 μg/ml or less, such as 06 μg/ml or 0.5 μg/ml.

Additionally or alternatively, the method of producing the lentiviral particle or the method of improving the infectious lentiviral titre using the closed linear DNA transfer vector may be optimised by altering the ratios between the various DNA constructs. Thus, the construct ratios may be altered such that an enhanced production of infectious lentiviral particles is achieved.

When a packaging cell is transfected with 4 DNA constructs (the closed linear transfer vector, GAG/POL vector, REV vector and ENV (or VSVg) vector), any appropriate construct molar ratio may be used. However, to gain an optimised infectious titres, the construct molar ratio is preferably (written as transfer:GAG/POL:REV:ENV DNA constructs) 4:1:2:1, 3:1:3:2, 3:1:3:1.5, or 3:1:2:1. The construct ratio may be any suitable ratio that falls between these ratios, such as 3:1:2.5:1 and the like. Preferably, the construct ratio is 4:1:2:1.

There is also provided a cell transfected with the closed linear transfer vector according to the first aspect of the invention. Said cell may be a packaging or producer cell as defined further herein. Further, the cell may also be transfected with one or more production vectors as described herein.

Further embodiments are described below and in the claims. Further advantages are described below.

### BRIEF DESCRIPTION OF THE DRAWINGS

FIG. 1A depicts gene expression in packaging cells 72h post transfection with a standard EF1α-eGFP-WPRE transfer vector, and lentiviral production vectors. RNA extracted from cells was subjected to RT-qPCR using the probes LTR-P and eGFP to quantify full length genomic RNA transcripts and total RNA transcripts derived from the transfer vector, respectively. Transfer vector gene expression from a standard closed linear DNA (dbDNA™) vector, without a poly(A) signal sequence and a spacer, was similar to corresponding plasmid DNA (pDNA), demonstrating that low infectious titres were not the result of insufficient transfer vector RNA.

FIG. 1B depicts DNA vector copies per cell measured by qPCR, demonstrating an excess of dbDNA vector copies relative to pDNA. The cells were transfected with 1 μg/ml total DNA. A construct mass ratio of 2:1:1:1 was used for pDNA, and the molar equivalent was used for dbDNA. Measurements were taken 72 hours post transfection. Data for each of the vectors is shown, and a comparison of the levels for plasmid versus closed linear DNA is given.

FIG. 2A depicts the titres from production using a standard EF1α-eGFP-WPRE transfer vector, without a poly(A) sequence and a spacer. This is a plot of construct vs titre. Total viral particle titre (p24) was five times lower for closed linear DNA (dbDNA™) transfection than for the corresponding pDNA transfection. However, the infectious viral titre for the dbDNA™ transfection was below the limit of detection (LOD), demonstrating that in this instance very few infectious viral particles are produced. Key: VP/mL is viral particle per millilitre and TU/mL is transducing units per millilitre (infectious titre).

FIG. 2B depicts total viral particle titre (VP/mL) in HEK293F producer cells transfected with decreasing total input standard closed linear vector at the indicated ratios of DNA:PEI

FIG. 3 depicts the titres from productions using a standard EF1α-eGFP-WPRE transfer vector whilst attempting to optimise conditions to improve infectious titre (plot of titre versus construct). 1 μg/mL plasmid constructs were transfected at a mass ratio of 2:1:1:1 (eGFP:Gagpol:Rev:VSVg), and 0.5 μg/mL closed linear dbDNA™ was transfected using the molar equivalent (Mol), or the indicated ratios. Optimising conditions for dbDNA™ transfection resulted in a complete rescue of total particle titres (p24), but infectious titres remained 100 times lower for standard closed linear DNA transfer vector than plasmid.

The results shown on FIGS. 4A and 4B (plot of titre vs construct) indicate that increasing the ratio of transfer vector does not rescue infectious titre (infectious titre—FIG. 4A). The results from the genomic titre assay (FIG. 4B) suggest that packaging of viral genomes into particles is inefficient for closed linear DNA vector compared to pDNA, and increasing the amount of transfer vector does not improve this situation.

FIG. 5 depicts various closed linear DNA vector architectures as used in the Examples. LV-eGFP includes a restriction enzyme recognition sequence for the enzyme Avrll 3′ to the 3′ SIN LTR. LV-eGFP-pA includes a SV40 late poly(A) signal sequence 3′ to the sequence for the 3′ SIN LTR. LV-eGFP-pA-FTS includes a SV40 late poly(A) signal sequence and a F region termination sequence 3′ of the sequence for the 3′ SIN LTR. LV-eGFP-pA-RS1 includes a SV40 late poly(A) signal sequence and a 1kb random spacer (RS) 3′ of the sequence for the 3′ SIN LTR. RSV1-LV-eGFP-pA-RS1 also includes a 1kb 5′ random spacer (RS) spacer. Other elements are the sequences for the 5′LTR, EF1α promoter, eGFP transgene, Random Spacer (RS), and WPRE element.

FIG. 6 (A-C) shows the effect on the total titre (6A), infectious titre (6B) and genomic titre (6C) of the different transfer vectors as shown in FIG. 5. Plots are construct vs titre. In this instance, LV-eGFP was compared to LV-eGFP-pA. Additionally, in the context of closed linear DNA vector only, the LV-eGFP was first digested using Avrll restriction enzyme to cleave off sequence downstream of the 3′ SIN LTR and mitigate putative read-though interference. Addition of the SV40 late poly(A) sequence improved infectious and genomic titres for both plasmid and closed linear DNA productions.

FIG. 7 (A-C) shows the effect on the total titre (7A), infectious titre (7B) and genomic titre (7C) of the different constructs as shown in FIG. 5. Plots are construct vs titre. Addition of the F region termination sequence or the 1kb random spacer sequence downstream of the SV40 late poly(A) sequence further boosts infectious and genomic titres in the context of closed linear DNA.

FIG. 8 (A-B) demonstrates the further optimisation of transfection conditions for closed linear DNA vectors in relation to infectious titre when compared to pDNA, which resulted in an infectious titre only two-fold lower than plasmid. FIG. 8A shows the effect of reducing total input closed linear DNA on infectious titre relative to the plasmid control, demonstrating peak titres at 0.7 μg/mL. FIG. 8B shows particle and infectious titres for productions using LV-eGFP-pA-RS1kb and optimised conditions.

FIG. 9 (A-D) depicts the plasmid map for (A) proTLx transfer vector, (B) proTLx Gag-Pol production vector, (C) proTLx Rev production vector and (D) proTLx VSVg production vector used in the Examples. (A) proTLx-K LV-eGFP-pA-RS1 includes a SV40 late poly(A) signal sequence 3′ to the sequence for the 3′ SIN LTR, and a 1kb random spacer (RS) 3′ of the sequence for the SV40 late poly(A) signal sequence. Other elements include the sequences for 5′ modified LTR, Random Spacer (RS), HIV-1 Psi, Rev Response Element (RRE), cPPT, EF1α promoter, eGFP transgene, WPRE, 5′TeIRL (protelomerase recognition site), Kanamycin resistance (KanR) promoter, KanR gene, and pUC ori. (B) proTLx Gag-Pol production vector includes the Gag and Pol genes. Other elements include the sequences for Random Spacer (RS), cPPT, RRE, beta-globin poly(A) signal sequence, Kanamycin resistance (KanR) promoter, KanR gene, and pUC ori and 5′TeIRL (protelomerase recognition site). (C) proTLx Rev production vector includes the Rev gene. Other elements include the sequences for 3′TeIRL (protelomerase recognition site), Random Spacer (RS), RSV promoter, HIV LTR poly(A) signal sequence, Kanamycin resistance (KanR) promoter, KanR gene, and pUC ori. (D) proTLx VSVg production vector includes the VSVg envelope protein gene. Other elements include the sequences for 3′TeIRL (protelomerase recognition site), Random Spacer (RS), CMV enhancer and promoter, beta-globin intron, beta-globin poly(A) signal sequence, Kanamycin resistance (KanR) promoter, KanR gene, and pUC ori.

FIG. 10 (A-C) demonstrates the further optimisation of transfection conditions in relation to infectious titre using the LV-RS1-eGFP-pA-RS1 transfer vector. FIG. 10A shows infectious titres comparing linear close-ended DNA LV-eGFP-pA-RS1 and LV-RS1-eGFP-pA-RS1, demonstrating a 1.8 fold improvement with the 3′ RS1. FIG. 10B depicts the infectious titre of LV produced using 0.7 μg/ml DNA and the indicated molar construct ratios. LV produced using 1 μg/mL plasmid DNA at a mass ratio of 2:1:1:1 (eGFP:GagPol:Rev:VSVg) was used as a control. FIG. 10C shows infectious titres using LV-RS1-eGFP-pA-RS1 at low DNA input, relative to plasmid. Closed linear DNA and plasmid DNA were transfected at a molar ratio of 4:1:2:1 and a mass ratio of 2:1:1:1, respectively.

FIG. 11A-B depicts the evaluation of 3′ RS1kb in production constructs, leading to rescue of closed linear DNA-derived LV. FIG. 11A shows the infectious titre of LV produced using 0.7 μg/mL dbDNA at a molar construct ratio of 4:1:2:1. The LV-RS1-eGFP-pA-RS1 transfer vector was used in combination with our standard accessory constructs (Std), and each production construct was iteratively swapped for the equivalent construct containing a 3′ RS1 element such that each construct was tested independently and in combination with all others. FIG. 11B shows the infectious titre of LV produced using a CAR19h28z transfer vector, GagPol-RS1, Rev-RS1, and VSVg (0.7 μg/mL dbDNA at a molar ratio of 4:1:2:1). Error bars represent the standard deviation between replicates.

### DETAILED DESCRIPTION OF THE INVENTION

The present invention relates to a novel closed linear DNA vector, which is suitable for use in the production of lentiviral particles. Most notably, this is suitable to produce a higher lentiviral infectious particle titre than closed linear DNA vectors lacking such a construction.

Lentiviral Particles and Their Production Lentiviral particles (LVPs) are a well-studied vector system based on human immunodeficiency virus (HIV-1). Other lentiviral systems have also been developed as gene transfer systems, including HIV-2, simian immunodeficiency virus (SIM), and non-primate lentiviruses such as feline immunodeficiency virus (FIV), equine infectious anaemia virus (EIAV), and caprine arthritis-encephalitis virus (CAEV). The lentiviral components useful for the production of a lentiviral particle are known in the art. See for example Zufferey et al. (1997) Multiply attenuated lentiviral vector achieves efficient gene delivery in vivo, Nature Biotechnology, 15:871-875 and Dull et al. (1998) A third-generation lentivirus vector with a conditional packaging system, Journal of Virology, 72(11):8463-8471 and Table 1.

TABLE 1
Cis-acting and Trans-acting elements of HIV-1 based lentiviral vectors

| | Function | Necessity |
|---|---|---|
| Cis-acting Element | | |
| LTRs | Contain sequences required for viral gene | Essential |
| | expression, reverse transcription, and integration. | |
| Psi (ψ) | Required for packaging of the genomic transfer RNA. | Essential |
| Rev Response Element | Sequence to which Rev protein binds. Required for | Beneficial |
| (RRE) | processing and transporting of viral RNAs. | |
| Central Polypurine Tract | Recognition site for proviral DNA synthesis. | Beneficial |
| (cPPT) | Increases transduction efficiency and transgene | |
| | expression. | |
| Trans-acting Element | | |
| GAG | Encodes structural proteins required for lentiviral | Essential |
| | particle production. | |
| POL | Encodes enzymes such as reverse transcriptase and | Essential |
| | integrase required for lentiviral particle production. | |
| ENV | Encodes envelope glycoprotein | Essential |
| VIF | Assists assembly of the virions and infectivity. | Optional |
| VPU | Assists release of virions. | Optional |
| VPR | Assists infection of non-dividing cells. | Optional |
| TAT | Binds TAR to activate transcription from the LTR | Optional |
| | promoter. Necessary for high-level expression of | |
| | viral LTR. | |
| REV | Binds to RRE within unspliced and partially spliced | Beneficial |
| | transcripts to facilitate nuclear export. | |
| NEF | Required for high viral burden. | Optional |

Guided by safety concerns due to the pathogenic nature of HIV-1 in humans, various generations of lentiviral systems have been developed for the production of lentiviral particles. For a summary of lentiviral systems that can be used for lentiviral particle production see Schweizer and Merten, 2010, Current Gene Therapy 10(6), 474-486; and Merten, Hebben and Bovolenta, 2016, Molecular Therapy—Methods & Clinical Development 3, 16017; doi:10.1038/mtm.2016.17. The most widely used lentiviral system for use in clinical and research and development purposes is the third-generation four-vector system that expresses:
1) Lentiviral group specific antigen (GAG) gene and a lentiviral polymerase (POL) protein
2) Envelope protein (usually Vesicular Stomatitis Virus Glycoprotein (VSV-G))
3) HIV regulator of expression of virion proteins (Rev) protein; and
4) A Transfer Vector containing a transgene or other coding sequence

Typically, the above-described four DNA vectors are in the form of plasmids. Traditionally, packaging cells, such as human embryonic kidney cells (e.g. HEK293) are transfected with each of the four plasmids as an adherent cell culture. The transiently transfected cells are able to produce lentiviral particles carrying the gene of interest.

However, as interest in suspension culture increases, HEK293F are becoming more widely used since suspension cultures are more amenable to commercial scale up.

The present invention relates to a novel closed linear DNA vector, which is suitable for use in the production of lentiviral particles according to any appropriate method.

### Closed Linear DNA Vector

The inventors have developed a novel closed linear DNA vector, also referred to as a ‘closed linear transfer vector’, which has certain features that enable it to outperform existing closed linear DNA vectors in the production of infectious titres of lentiviral particles. The closed linear DNA vector may take any appropriate form, with any type of ‘closed’ ends. A closed linear DNA vector may also be referred to as a closed linear DNA molecule.

Closed linear DNA is generally understood to be double-stranded DNA covalently closed or capped at each end. The double stranded section, or duplex part of the DNA, is therefore complementary. When denatured, closed linear DNA may form a single stranded circle. The DNA may be closed at each end by any suitable structure, including a cruciform, a hairpin, or a hairpin loop, depending on preference. The end of the closed linear DNA may be composed of a non-complementary sequence, thus forcing the DNA into a single stranded configuration at the cruciform, hairpin, or hairpin loop. Alternatively, the sequence can be complementary such that the end forms a hairpin.

It may be preferred that the end is formed by a portion of a target sequence for a protelomerase enzyme. A protelomerase target sequence is any DNA sequence whose presence in a DNA template allows for the enzymatic activity of protelomerase, which cuts a double stranded section of DNA and re-ligates them, leaving covalently closed ends. In general, a protelomerase target sequence comprises any perfect palindromic sequence i.e. any double-stranded DNA sequence having two-fold rotational symmetry, or a perfect inverted repeat. The closed linear DNA may have a portion of a protelomerase target sequence at one or both ends. The protelomerase target sequence can be for the same cognate protelomerase at each end, or be the cognate sequence for a different protelomerase for each end. Closed linear DNA constructed via the action of various protelomerase enzymes have been previously disclosed by the applicants in WO2010/086626, WO2012/017210, WO2016/132129 and WO2018/033730, all of which are incorporated by reference. Closed linear DNA constructed using in vitro DNA amplification followed by cleavage with a protelomerase enzyme has the advantage that the closed linear DNA is produced in an in vitro, cell-free environment, and can be scaled up for commercial production. These closed linear DNA vectors are known as Doggybone™ DNA or dbDNA™. It may be preferred that the closed linear DNA vectors are made using the prior methods of the applicants, in an in vitro, cell-free manner based upon polymerase based amplification of a DNA template with at least one protelomerase target sequence, and processing of the amplified DNA with a protelomerase to produce closed linear DNA.

Closed linear DNA can be constructed by a conversion of a plasmid with the requisite protelomerase target sequences into a closed linear DNA vector, although this is not an efficient method of production.

Other closed linear DNA vectors have been constructed by various in vitro strategies including the capping of PCR products, and the “minimalistic immunogenic defined gene expression (MIDGE)” vectors. MIDGE is generated by the digestion of both prokaryotic and eukaryotic backbones after isolation of plasmid from bacterial cells, followed by ligation of the required DNA sequence into hairpin sequences for end-refilling. Structures made by such methods would also be suitable for the DNA vectors of the present invention.

DNA “ministrings”, which are produced in an in vivo manner in cell culture, based upon the action of protelomerase, are also closed linear DNA vectors that would be suitable for use in the invention.

Other forms of closed linear DNA that may be suitable include those closed at the ends with cruciform structures, which can again be manufactured in cell culture or in vitro enzymatically.

It may be preferred that the closed linear DNA is manufactured in a cell-free system, since this ensures purity of product; in the alternative, stringent purification of closed linear DNA made by cellular methods will be required by the regulatory authorities.

Closed linear DNA vectors can be designed to be minimal vectors, including only the sequences necessary for their desired function and structure (i.e. the sequence they are delivering and a sequence encoding the closed ends, for example a cruciform, hairpin or hairpin loops at the end of the double stranded linear section). Unnecessary or extraneous sequences (such as bacterial sequences) that may be excluded from closed linear DNA vectors may include bacterial origins of replication, bacterial selection markers (e.g. antibiotic resistance genes), and unmethylated CpG dinucleotides. The non-inclusion of such sequences, enables the creation of a “minimal” vector which does not contain extraneous genetic material. This may be preferred where the cells are to be used for therapeutic purposes, since no genetic material is introduced that could affect the performance of the vector or cause unnecessary side effects (i.e. antibiotic resistance genes).

The applicants have previously used closed linear DNA vectors in the manufacture of “second generation” lentiviral vectors. Such closed linear DNA vectors did not include any further modifications when compared to the pDNA vectors commonly used. Such unmodified closed linear DNA vectors as described above are inefficient transfer vectors for the production of lentiviral vectors, particularly for third generation lentiviral methods, as demonstrated in Example 1. The inventors have thus developed a novel closed linear DNA vector, herein referred to as a ‘closed linear transfer vector’, which has designed features that enable it to outperform existing closed linear DNA vectors in the production of infectious titres of lentiviral particles.

In one aspect, the present invention relates to a closed linear DNA vector suitable for use as a lentiviral transfer vector, said closed linear DNA vector comprises sequences in the following order 5′ to 3′
(a) a hybrid 5′ long terminal repeat (LTR) sequence;
(b) a promoter operably linked to a transgene;
(c) a 3′ self-inactivating (SIN) LTR sequence;
(d) a poly(A) signal sequence; and
(e) a spacer sequence.

Additional sequences may be included within the closed linear DNA vector, for example additional spacer sequences.

In the vector, the promoter and transgene, together with any additional sequences for inclusion into the particle, are effectively flanked by the 5′ and 3′ LTR sequences. As used herein, flanked or flanking does not mean that the 5′LTR and 3′SIN LTR have to be immediately adjacent to the promoter and transgene, but instead provide the ends of the RNA molecule to be packaged into the LV particle. It will be understood by those skilled in the art that the LTR sequences from the “flanking” ends of the single stranded RNA for packaging into the LV particle. In retroviruses, The LTR-flanked sequences are partially transcribed into an RNA intermediate, followed by reverse transcription into complementary DNA (cDNA) and ultimately dsDNA (double-stranded DNA) with full LTRs. The LTRs then mediate integration of the DNA via an LTR specific integrase into another region of the host chromosome. In one aspect, the present invention thus relates to a novel closed linear DNA vector, herein referred to as a ‘closed linear transfer vector’ comprising:
a) a promoter operably linked to a transgene;
b) a sequence encoding a hybrid 5′ long terminal repeat (LTR), and a sequence encoding 3′ SIN LTR flanking the promoter and transgene;
c) a sequence encoding a poly(A) signal located 3′ to the 3′LTR; and
d) a spacer sequence located 3′ to the sequence encoding a poly(A) signal.

This closed linear transfer vector is suitable for use as a transfer vector for the manufacture of infectious lentiviral particles (lentiviral vector).

In particular, the features (c) a sequence encoding a poly(A) signal and (d) a spacer sequence provide the novel closed linear DNA vector with characteristics that enhance the production of infectious titres of lentiviral particles. Such infectious particles contain the transgene. These features are described in more detail below.

As explained previously, the sequences in the closed linear DNA transfer vector may be described as sequences coding for elements within an RNA molecule or may be described as sequences for those elements perse. It will also be understood, that as the closed linear DNA is a duplex both the relevant sequence element and its complementary sequence will be present in the vector.

### Poly(A) Signal Sequence

The polyadenylation process is generally required for the synthesis of messenger RNA (mRNA), in which RNA cleavage is coupled with synthesis of polyadenosine monophosphate (adenine base) on the newly formed 3′ end of the RNA. The sequence elements for polyadenylation include the polyadenylation signal (poly(A) signal) in the RNA sequence. In mRNA, the added stretch of polyadenosine monophosphate is called the polyadenylation tail (poly(A) tail). The poly(A) tail can contribute to increased translational efficiency.

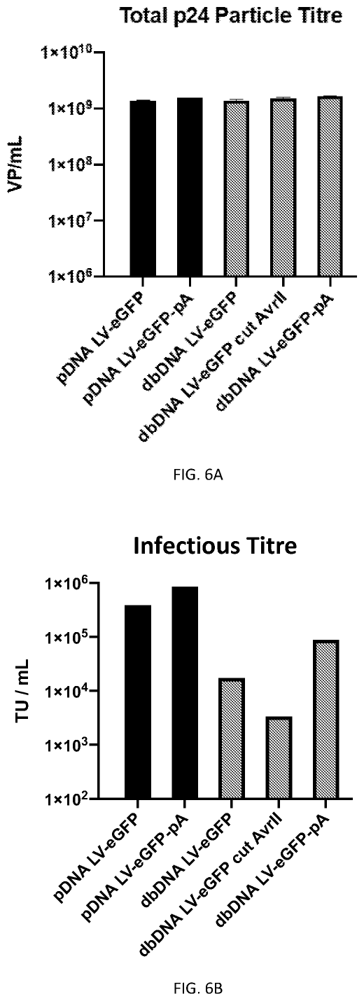

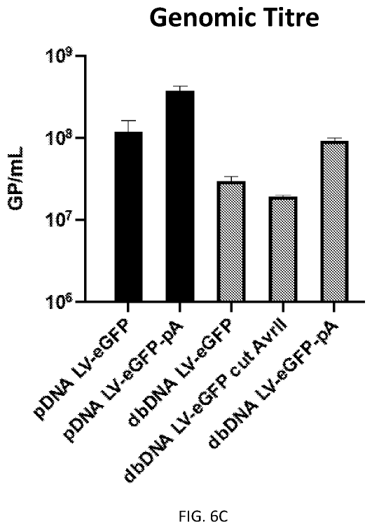

The inventors have found that including a sequence encoding a polyadenylation (poly(A)) signal (or “poly(A) signal sequence”) 3′ to the sequence encoding the 3′ LTR in a closed linear DNA molecule for use as a lentiviral transfer vector increased both genomic and infectious viral titres (see Example 1 and FIG. 6). Poly(A) signal sequences may be found at the 3′ of eukaryotic protein-encoding genes. Generally, the central sequence motif AAUAAA or AUUAAA is a key element of the poly(A) signal in RNA, and this central sequence may require flanking, auxiliary elements for both 3′-end cleavage and polyadenylation of pre-messenger RNA as well as to promote downstream transcriptional termination. A number of poly(A) signals are known in the art, and any appropriate sequence may be used. Poly(A) signals in RNA may include the sequence AAUAAA, include at least one sequence that is GU rich, and/or at least one sequence that is U rich.

Preferably, the sequence encoding a poly(A) signal is encoding a strong poly(A) signal. Thus, a strong poly(A) signal sequence is preferred. A number of strong poly(A) signals are known in the art, and these may be defined as providing an efficient termination of transcription. Transcription termination is the process whereby the transcription complex and nascent RNA are both released from the template DNA. The skilled person will be able to determine using routine methods whether a poly(A) signal provides for efficient termination. In a preferred embodiment, the sequence encoding a strong poly(A) signal is selected from the SV40 Late polyA sequence, or a sequence having at least 90% homology thereto, rabbit P-globin (rbGlob) poly(A) sequence, or a sequence having at least 90% homology thereto, or bovine growth hormone poly(A) (bGHpA), or a sequence having at least 90% homology thereto. Such may be described as “strong” poly(A) signals.

The sequence encoding a poly(A) signal may further comprise an upstream sequence element (USE). USEs are well known in the art, and are thought to improve efficiency of the polyadenylation signal.

### Spacer Sequence

### 3′Spacer Sequence

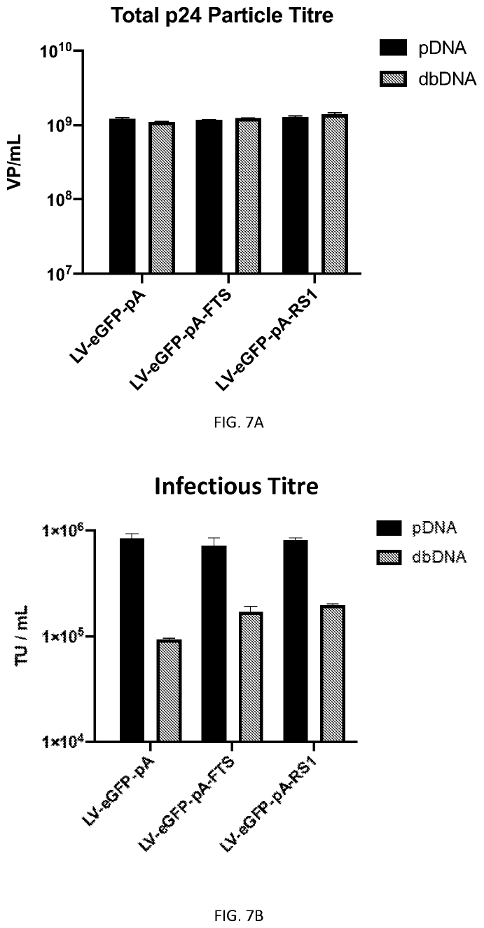

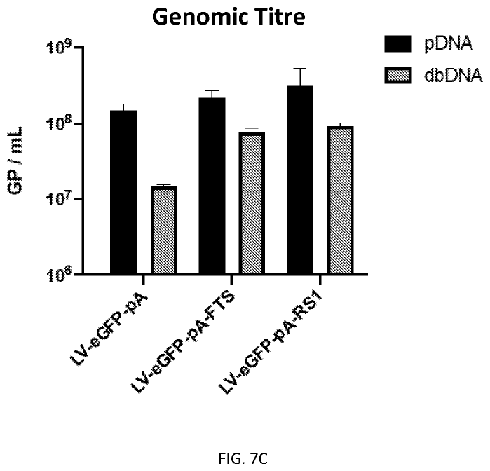

The inventors have found that including a spacer sequence 3′ of the sequence encoding a poly(A) signal in the novel closed linear DNA vector described above further increased both genomic and infectious viral titres (see Example 1 and FIG. 7). The spacer sequence 3′ of the sequence encoding a poly(A) signal (poly(A) signal sequence) in the novel closed linear DNA vector may be referred to as the downstream spacer sequence, or 3′ spacer sequence. Interestingly, incorporation of the same spacer sequence into a plasmid-based lentiviral transfer vector did not affect viral titres (see Example 1 and FIG. 7), and thus the effect is thought to be dependent upon the format of the closed linear vector itself. The spacer sequence may be any suitable length and any suitable sequence. Preferably, the spacer sequence of the novel closed linear transfer vector may be least 250 nucleotides in length. The spacer may be at least 100, at least 200, at least 300, at least 400, at least 500, at least 600, at least 700 or at least 800, at least 900, at least 1000, at least 1100, at least 1200, at least 1300, at least 1400, or at least 1500 nucleotides in length. It may be preferred that the spacer is at least 250, at least 500 or most preferably at least 1000 nucleotides in length (1kb). The spacer separates the sequence encoding the poly(A) signal from the closed end of the linear DNA molecule. If the end is closed with a portion of a protelomerase sequence, the 3′ end of the spacer sequence may be adjacent to the 5′ end of the portion of the protelomerase sequence. If the end is closed with a hairpin, the same concept may apply, the sequences for the hairpin and spacer may be adjacent. The spacer sequence may be of any appropriate length. Since this is not present in the final lentiviral vector (infectious lentiviral particle), the capacity of the lentiviral vector genome does not require consideration in determining the length of the spacer sequence.

As the spacer sequence is present in the duplex DNA, the spacer sequence may be determined in terms of base pairs in length. The spacer sequence is optionally non-coding DNA, for example, it does not code for a protein or RNA product. The sequence of the spacer may be random. Without wishing to be bound by theory, the inventors postulate that the spacer sequence facilitates efficient RNA processing when the transfer vector is in the form of a closed linear DNA. The inventors have noted that this is a specific requirement due to the architecture of the closed linear DNA, and that addition of a spacer sequence in plasmid DNA made no difference to infectious titre (see Example 1 and FIG. 7).

### 5′ Spacer Sequence

The inventors have found that including a spacer sequence 5′ of the 5′ long terminal repeat (5′LTR) in the novel closed linear DNA molecule described above further improved infectious viral titres (see Example 2 and FIG. 7B). The spacer sequence 5′ of the 5′LTR in the novel closed linear DNA molecule may be referred to as the upstream spacer sequence. The spacer sequence may be any suitable length and any suitable sequence. Preferably, the sequence of the downstream spacer is not identical to the sequence of the upstream spacer. Preferably, the sequence of the downstream spacer may be different to the sequence of the upstream spacer. Preferably, the spacer sequence of the novel closed linear transfer vector may be at least 250 nucleotides in length. The spacer may be at least 100, at least 200, at least 300, at least 400, at least 500, at least 600, at least 700 or at least 800, at least 900, at least 1000, at least 1100, at least 1200, at least 1300, at least 1400, or at least 1500 nucleotides in length. Preferably, the spacer is at least 1kb in length. The spacer separates the 5′LTR from the closed end of the linear DNA molecule. If the end is closed with a portion of a protelomerase sequence, the 5′ end of the upstream spacer sequence may be adjacent to the 3′ end of the portion of the protelomerase sequence. If the end is closed with a hairpin, the same concept may apply, the sequences for the hairpin and spacer may be adjacent. The upstream spacer sequence may be of any appropriate length. Since this is not present in the final lentiviral vector (infectious lentiviral particle), the capacity of the lentiviral vector genome does not require consideration in determining the length of the upstream spacer sequence.

As the upstream spacer sequence is present in the duplex DNA, the upstream spacer sequence may be determined in terms of base pairs in length. The upstream spacer sequence is optionally non-coding DNA, for example, it does not code for a protein or RNA product. The sequence of the upstream spacer may be random. The sequence may also be different to any downstream spacer sequence.

### Transgene

The closed linear transfer vector of any aspect of the invention may comprise an expression cassette comprising, consisting or consisting essentially of a eukaryotic promoter operably linked to a sequence encoding a product of interest. The sequence encoding a product of interest may be referred to as a transgene. The transgene may encode an RNA product, such as an inhibitory RNA (for example, microRNA, or small hairpin RNA (shRNA)) or a protein product (via messenger RNA). The closed linear transfer vector of any aspect of the invention preferably includes a promoter or enhancer operably linked to a transgene. One or more promoter or enhancers may be used, as required. Any suitable promoters or enhancers can be used. These are for the expression of the transgene once the Lentiviral vector has been constructed and applied to the cell which it is desired to target.

The transgene selected will depend on the specific use intended for the lentiviral vector. Illustrative, non-limiting, examples of transgenes include a transgene encoding a therapeutic RNA (e.g. a transgene encoding an antisense RNA complementary to a target RNA or DNA sequence), a gene therapy transgene encoding a protein defective or absent in a diseased subject, and a vaccine transgene used for DNA vaccination (i.e. encoding a protein the expression of which will induce vaccination of the recipient organism against said protein).

A “promoter” is a nucleotide sequence which initiates and regulates transcription of a polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “enhancer” includes full-length promoter regions and functional (e.g., controls transcription or translation) segments of these regions. The term includes bidirectional promoters.

In the examples, EF1α (Elongation Factor 1-Alpha) is used as a promoter. This is a constitutive promoter and may therefore be desirable for use in expressing the transgene in the target cell once the Lentiviral vector has been delivered. Modified EF1α promoters may also be of use, such as the hEF1α-HTLV promoter, which is a composite promoter comprising the human EF1α core promoter and the R segment and part of the U5 sequence (R-U5′) of the Human T-Cell Leukaemia Virus (HTLV) Type 1 Long Terminal Repeat. The EF1α promoter exhibits a strong activity and yields long lasting expression of a transgene in vivo. The R-U5′ has been coupled to the core promoter to enhance stability of RNA. Alternative promoters suitable for use in the present invention include but are not limited to: cytomegalovirus (CMV) promoter, murine stem cell virus (MSCV) promoter, phosphoglycerate kinase 1 (PGK) promoter, thymidine kinase (TK) promoter, spleen focus forming virus (SFFV) promoter, CAG promoter and polyubiquitin C (UBC) promoter or transcriptionally active fragment thereof. The promoter may also be chosen to permit a cell-specific expression once the lentiviral vector has been administered in vivo. For example, targeting to melanoma cells has been achieved by including the tyrosinase promoter or enhancer fragment. A person skilled in the art will be able to select a suitable cell-specific promoter for use in the present invention.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence. Thus, the term “operably linked” is intended to encompass any spacing or orientation of the promoter element and the transgene which allows for initiation of transcription of the transgene upon recognition of the promoter element by a transcription complex in vivo.

In a particular embodiment, a multicistronic expression cassette may be used in the closed linear transfer vector. A multicistronic expression cassette comprises multiple genes operably linked to a single promoter in a single expression cassette, enabling translation of multiple genes from a single transcript. Multicistronic expression cassettes may be desirable, as they enable selectable agents or marker genes to be co-expressed, permit manageable construct size, enable constant production of desired gene products and provide an opportunity to include a conditional cytotoxic gene as a failsafe for cases where an adverse clinical event may occur. Methods to design and produce functional multicistronic expression cassettes are well known in the art, and include use of internal ribosome entry site (IRES), self-cleaving 2A peptides, and/or bidirectional promoters. See for example Shaimardanova et al (2019) Production and Application of Multicistronic Constructs for Various Human Disease Therapies. Pharmaceutics, 11(11):580, doi:10.3390/pharmaceutics11110580 and Golding, M. & Mann, M. (2011) A bidirectional promoter architecture enhances lentiviral transgenesis in embryonic and extraembryonic stem cells. Gene Therapy, 18:817-826, https://doi.org/10.1038/gt.2011.26.

LTR sequences

In any embodiment of the present invention, the closed linear transfer vector comprises sequences encoding a 5′ long terminal repeat (LTR) and a 3′ LTR flanking the promoter and transgene. Alternatively put, the vector includes a 5′ long terminal repeat (LTR) sequence and a 3′ LTR sequence flanking the promoter and transgene. The order is thus: 5′LTR; promoter operably linked to a transgene; 3′LTR. Additional sequences may be included between the LTRs. Additional sequences may be present in the vector outside of the sequence flanked by the LTRs.

The LTRs are virally-derived elements that facilitate integration of the transgene into the host cell's genome. Wild-type LTRs comprise a Unique 3′ (U3) region, a Repeat (R) region, and a Unique 5′ (U5) region, such that wild-type 5′ LTR and 3′ LTR both have a U3-R-U5 structure. In third-generation lentiviral particle platforms, the sequences encoding the LTRs are modified compared to wild-type lentiviral LTRs, in order to make lentiviral-based vectors safer for use in research and clinical settings.

The LTR sequences used in the present invention may be derived from any lentivirus. Lentivirus is a genus of retroviruses that includes human immunodeficiency virus (HIV—types 1 to 3). Lentiviral vectors may be derived from primate lentiviruses (HIV-2 and Simian immunodeficiency virus (SIV)) and non-primate lentiviruses (such as Maedi Visna virus (MVV), Feline immunodeficiency virus (FIV), Equine Infectious Anaemia Virus (EIAV), Caprine arthritis encephalitis virus (CAEV), Jembrana disease virus (JDV), Puma lentivirus, lion lentivirus, and Bovine immunodeficiency virus (BIV)), although HIV-based vectors make up the majority of lentiviral vectors in current use. The LTRs may therefore be derived from any lentivirus, but is preferably derived from HIV-1.

In any aspect of the invention, the 5′ LTR is a hybrid LTR (may also be referred to as a modified 5′ LTR). A hybrid LTR indicates that a portion of the wild type LTR has been removed, and a heterologous sequence has been inserted. The hybrid 5′ LTR may permit Tat-independent transcription. In order to reduce or remove the dependence on Tat, all or part of the U3 region may be deleted. In order to maintain expression, the function of the U3 region may be replaced using a heterologous promoter. Such a promoter may be another viral promoter, for example the cytomegalovirus (CMV) promoter.

Any suitable sequence for a hybrid 5′ LTR can be used in the present invention, and several are known in the art. A hybrid 5′ LTR is not a wild-type viral LTR.

In a preferred embodiment, the sequence encoding the 5′ LTR is partially deleted and fused to heterologous enhancer or promoter elements, to enable Tat-independent expression of the transgene. Thus the 5′LTR sequence is thus partially deleted and fused to heterologous enhancer or promoter elements.

In a preferred embodiment, the sequence encoding the 3′ LTR is a sequence for a 3′ self-inactivating (SIN) LTR. Alternatively put, the vector includes a 3′ SIN LTR sequence A 3′ SIN LTR has one or more deletions compared to a wild-type lentiviral 3′ LTR, and may be referred to a modified 3′ LTR. The one or more deletions are transferred into the 5′LTR after one round of reverse transcription. This deletion abolishes transcription of the full-length virus after it has incorporated into a host cell. The one or more deletions may include partial or complete deletion of promoter or enhancer elements including the TATA box and binding sites for transcription factors Sp1 and NF-κB. 3′ SIN LTRs are well known in the art, and a skilled person will be able to identify appropriate constructs. In a preferred embodiment the 3′ SIN LTR comprises a 133 nucleotide deletion in the U3 region of the 3′ LTR, at nucleotide position −149 to −9 with respect to the transcription start site of a wild-type lentiviral 3′ LTR. A SIN 3′ LTR is not a wild-type viral LTR.

Further to the deletions in the 3′ LTR, the 3′ SIN LTR may include heterologous sequences to impart a particular function. Thus, the 3′ LTR may also be described as a hybrid LTR. Any heterologous sequence elements may be inserted into the 3′LTR. For example, heterologous regulatory elements may be inserted. Any suitable sequence encoding a hybrid SIN 3′ LTR can be used in the present invention, and several are known in the art. A hybrid SIN 3′ LTR is not a wild-type viral LTR.

Further, the 3′ SIN LTR may comprise a USE-element in the place of the deletion of the U3 region. Preferably, the USE-element is derived from SV40.

The sequences included in the closed linear DNA vector of the present invention are preferably those encoding LTRs derived from HIV-1, but it will be clear that similar modifications can be applied to other suitable LTRS to have a similar effect.

### Including Sequences Encoding Other Elements

The closed linear transfer vector may comprise sequences encoding further elements, or sequences for additional elements, as summarised in Table 1. Such elements may include the RNA packaging signal Psi (ψ), which may usually be located 3′ to the 5′ LTR, the Rev Response Element (RRE), which may usually be located 3′ of Psi, and a central polypurine tract (cPPT), which may usually be located 3′ of the RRE. Further additional functional sequences may be encoded or included, such as a primer binding site (PBS) or a Woodchuck Hepatitis Post-Transcriptional Regulatory Element (WPRE), can also be advantageously included in the closed linear transfer vector of the present invention, to obtain a more stable expression of the transgene in vivo. WPRE can increase transgene expression from viral vectors, although the precise mechanism of action is not known. WPRE is most effective when placed downstream of the transgene, proximal to the polyadenylation signal. WPRE may be substituted for other post-transcriptional regulatory elements (PREs) from other viruses. WPRE is thought to reduce the transcriptional read-through from lentiviral 3′-LTRs, and is used in the present Examples. Given its presence in the closed linear DNA vectors originally tested (pre-modification) it was a surprise to the inventors that the performance of the closed linear lentiviral transfer vector could be improved by making the modifications described herein.

### Method of Making a Lentiviral Vector

In a second aspect of the invention, provided herein is a method of producing infectious lentiviral particles (LVP), also described as lentiviral vectors.

In embodiments, the methods described herein include transfecting a packaging cell with the closed linear transfer vector, described above, and one or more production vectors.

In embodiments, the methods described here include transfecting a production cell with the closed linear transfer vector, described above.

### Production Vectors

As used herein, the term ‘production vector’, or ‘production construct’, refers to a vector that contains the sequences encoding the components necessary to produce a lentiviral particle and ‘package’ a gene of interest (or transgene) in the final, infectious lentiviral particle. These may also be referred to as ‘packaging elements’ (in particular the GAG, POL or REV elements). The production vector includes an expression cassette, which refers to a distinct component of a vector, and includes one or more genes and regulatory sequences to be delivered into, and ultimately expressed by, a transfected packaging cell. One or more production vectors, each comprising one or more expression cassettes, may be transfected into a packaging cell. In the art, these may also be called “accessory constructs” or “helper constructs”.

The lentiviral regulator of expression of virion proteins (REV) gene encodes for an RNA-binding protein that binds to the Rev Response Element (RRE) within unspliced or partially spliced transcripts to facilitate their transport from the nucleus to the cytoplasm.

The envelope (ENV) gene encodes for an envelope protein that is essential for the produced lentiviral particle to gain host cell entry. The lentiviral particle can be a pseudotyped vector, comprising a modified envelope protein, an envelope protein derived from a different virus or a chimeric envelope protein, allowing transduction of host cells lacking CD4. A range of different envelope proteins can be used for the production of envelope pseudotyped lentiviral particles Accordingly, for example, the ENV gene can encode a Vesicular Stomatitis Virus Glycoprotein (VSV-G) protein, which binds LDL-Receptor family members, allowing the lentiviral particle to infect a wide range of cell types of many distinct host species, including a variety of human cells. Preferably, the ENV gene encodes for VSV-G.

Alternative envelope proteins may be selected by a person skilled in the art, including the envelope protein of nonhuman retroviruses such as the ecotropic retrovirus murine leukaemia virus (MULV), the gibbon ape leukaemia virus (GALV), the feline endogenous RD114 retrovirus, Moloney MULV 4070A, Moloney MULV strain 10A1, as well as the rabies virus glycoprotein, and the measles virus hemagglutinin and fusion glycoproteins.

The GAG gene encodes for a polyprotein that is translated from an unspliced mRNA which is then cleaved by the viral protease (PR) into the matrix protein, capsid, and nucleocapsid proteins. The lentiviral polymerase (POL) gene encodes the enzymatic proteins reverse transcriptase, protease, and integrase.

Each function (or component) can be derived from any suitable lentivirus. However, in a preferred embodiment, the GAG-POL and REV are derived from a HIV virus, in particular from HIV-1 or HIV-2.

At present, it is considered in the art that the optimal number of vectors supplied to a cell in total, from any source, is four. This optimal number appears to be necessary in order to minimise risk of viral propagation. However, in future it may be possible to produce lentiviral particles using more than, or less than four vectors. For example, using two, three, five or six vectors.

In a preferred embodiment, the packaging cell is transfected with the closed linear transfer vector and at least one production vector, each production vector comprising at least one expression cassette encoding one or more of:
1) Lentiviral group specific antigen (GAG);
2) Lentiviral polymerase (POL) protein;
3) Envelope protein (preferably Vesicular Stomatitis Virus Glycoprotein (VSV-G)); or
4) HIV regulator of expression of virion proteins (Rev) protein
   such that the transfected cell contains all the components necessary to produce a lentiviral particle.

A production vector may comprise more than one expression cassette. The GAG gene and POL gene may be included on a single production vector. GAG and POL may therefore share the same promoter sequence.

It should be noted, the ‘production vector’ is sometimes referred to as a ‘packaging vector’ in the art.

The production vectors may be provided to the cell in the form of a closed linear DNA vector or a circular DNA vector, such as a plasmid or minicircle. It may be preferred that all of the DNA vectors used are closed linear DNA, or a mixture of vector architectures may be used.

### Closed Linear Production Vector

Wherein a production vector is in the form of a closed linear DNA vector, it may take any appropriate form, with any type of ‘closed’ ends, as described above. Wherein a production vector is in the form of a closed linear DNA vector, it may be referred to as a closed linear production vector.

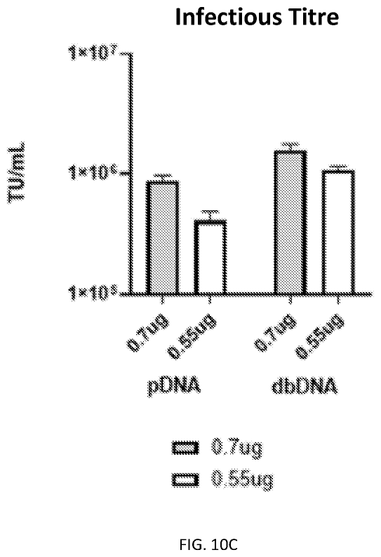

The inventors have found that inclusion of a spacer sequence 3′ of the expression cassette in the closed linear production vector provides improvements in infectious titres (see Examples 3 and 4, and FIGS. 10A and 12A).

Thus, the invention further relates to a closed linear DNA vector suitable for use as a production vector (closed linear production vector), said closed linear production vector comprising:
a) at least one expression cassette including one or more of:
   (i) a lentiviral group specific antigen (GAG) gene;
   (ii) a lentiviral polymerase (POL) gene; and/or
   (iii) an envelope gene (ENV); and/or
   (iv) a lentiviral regulatory gene (REV); and/or
b) a spacer sequence located 3′ to the expression cassette.

An expression cassette is a distinct component of vector DNA consisting of at least one gene and a regulatory sequence (such as a promoter) to be expressed by a transfected cell and a terminator element. The termination element may be any appropriate element, including a polyA sequence or indeed an LTR or modified LTR.

The spacer sequence 3′ of the expression cassette in the closed linear production vector may be any suitable length and any suitable sequence. Preferably, the spacer sequence of the closed linear production vector may be least 250 nucleotides in length. The spacer may be at least 100, at least 200, at least 300, at least 400, at least 500, at least 600, at least 700 or at least 800, at least 900, at least 1000, at least 1100, at least 1200, at least 1300, at least 1400, or at least 1500 nucleotides in length. It may be preferred that the spacer is at least 250, at least 500 or most preferably at least 1000 nucleotides in length (1kb). The spacer separates the expression cassette from the closed end of the linear DNA molecule. If the end is closed with a portion of a protelomerase sequence, the 3′ end of the spacer sequence may be adjacent to the 5′ end of the portion of the protelomerase sequence. If the end is closed with a hairpin, the same concept may apply, the sequences for the hairpin and spacer may be adjacent. The spacer sequence may be of any appropriate length. Since this is not present in the final lentiviral vector (infectious lentiviral particle), the capacity of the lentiviral vector genome does not require consideration in determining the length of the spacer sequence.

As the spacer sequence is present in the duplex DNA, the spacer sequence may be determined in terms of base pairs in length. The spacer sequence is optionally non-coding DNA, for example, it does not code for a protein or RNA product. The sequence of the spacer may be random. Without wishing to be bound by theory, the inventors postulate that the spacer sequence facilitates efficient RNA processing when the transfer vector is in the form of a closed linear DNA. It may be preferred that if two spacer sequences are present, that they are different sequences.

The one or more production vectors used may have the same vector architecture, or a mixture of vector architectures may be used. In other words, any combination of production vectors in the form of closed linear DNA vectors with or without a 3′ spacer sequence, or a circular DNA vector, such as a plasmid or minicircle, may be used.

It can be understood by an individual skilled in the art that the closed linear transfer vector of the present invention may further comprise any one or more of the above-described packaging elements, and/or the elements outlined in Table 1. These packaging elements may be present in the closed linear transfer vector as part of a multicistronic expression cassette, or as a separate expression cassette. For example, an expression cassette encoding the GAG gene may be included in the closed linear transfer vector.

### Packaging Cell

As used herein, the term ‘packaging cell’ refers to a cell for use in the production of lentiviral particles. Preferably, the packaging cell is a mammalian cell.

Mammalian cells for the production of lentiviral particles are known in the art. Representative examples of packaging cells include Human Embryonic Kidney (HEK) 293 cells and derivatives or variants thereof. For example, in some embodiments 293 variants may be selected for their ability to grow in suspension under serum-free conditions and which are ideally highly permissive to transfection. An example of such a variant is HEK293F cells. Alternatively, 293 variants may be selected for their ability to grow in adherent cell cultures, for example HEK293T cells. Other cell types for use as packaging cells include, but are not limited to, HeLa cells, A549 cells, KB cells, CKT1 cells, NIH/sT3 cells, Vero cells, Chinese Hamster Ovary (CHO) cells, or any eukaryotic cell which support the lentivirus life cycle.

The packaging cell may be constitutive or inducible.

The packaging cells can be cultured in a serum-free medium selected with respect to the specific cell used and permitting the production of the lentiviral particle. The serum-free medium allows production of lentiviral particle suitable for therapeutic applications. For a review on serum-free media, see Chapter 9 (Serum-Free Media) of Culture of Animal Cells: A Manual of Basic Technique; Ed. Freshen, RI, 2000, Wiley-Lisps, pp. 89-104 and 105-120. In general, serum free media will be manipulated to enhance growth of the respective cell line in culture, with a potential for inclusion of any of the following: a selection of secreted cellular proteins, diffusible nutrients, amino acids, organic and/or inorganic salts, vitamins, trace metals, sugars, and lipids as well as perhaps other compounds such as growth promoting substances (e.g., cytokines). Such media are commercially available, and the person skilled in the art will be able to select the appropriate media with respect to the mammalian host cells. The medium may be supplemented with additives such as a non-ionic surfactant such as Pluronic® F68 (Invitrogen, catalogue No. 24040-032), used for controlling shear forces in suspension cultures, an anti-clumping agent (e.g. from Invitrogen, catalogue No. 0010057AE) and L-glutamine or an alternative to L-glutamine such as a L-alanyl-L-glutamine dipeptide, e.g. GlutaMAX™ (Invitrogen, catalogue No 35050-038). The media and additives used in the present invention are advantageously GMP compliant. For example, a non-limiting example of a commercially available serum-free media which can be used for growing 293F cells in suspension is Gibco LV-MAX Production Media (ThermoFisher Scientific, catalogue No. A3583401).

Alternatively, the packaging cells can be cultured in an adherent system using methods well known in the art, see for example Merten et al. (2011) Large-Scale Manufacture and Characterization of a Lentiviral Vector Produced for Clinical Ex Vivo Gene Therapy Application. Human Gene Therapy, 22(3):343-356. http://doi.org/10.1089/hum.2010.060.

### Producer Cell

As used herein, the term ‘producer cell’ refers to a cell for use in the production of lentiviral particles. Producer cells are stable cell lines wherein all or part of the packaging functions required to produce an infectious lentiviral particle are inserted into the cellular genome, such that only the closed linear transfer vector is introduced via transient transfection. Such producer cells are known in the art, see for example U.S. Pat. No. 5,686,279, Ory et al. (1996) A stable human-derived packaging cell line for production of high titer retrovirus/vesicular stomatitis virus G pseudotypes. PNAS USA, 93:11400-11406 and Sanber et al. (2015) Construction of stable packaging cell lines for clinical lentiviral vector production. Sci Rep, 5:9021.

Producer cells can be constitutive or inducible, and are well known in the art (Farson et al. (2001) A new-generation stable inducible packaging cell line for lentiviral vectors. Hum Gene Ther, 12(8):981-97. doi: 10.1089/104303401750195935. and Merten, O. W., Hebben, M. & Bovolenta, C. (2016) Production of lentiviral vectors. Mol. Ther. Methods Clin. Dev. 3:16017.

Hybrid stable cell lines have also been developed wherein some packaging functions have been integrated into the cellular genome, whilst others are provided through transient transfection of packaging vectors. Thus, a combination of these procedures can be used, with some of the production vectors integrated into the cellular genome and others provided by transient transfection. The skilled person can appreciate that several different methods and reagents may be used to make infectious lentiviral particles.

Thus, it can be understood by someone skilled in the art that there are multiple strategies to produce lentiviral vectors using the novel closed linear transfer vector of the present invention. Overall, the packaging cell or producer cell to which the closed linear transfer vector is introduced should have all of the packaging functions necessary to produce a functional lentiviral particle, and these packaging functions may be introduced to the cell through transient transfection, stably integrated into the cellular genome, or a combination of the two.

### Transfection

In the method of the present invention, packaging cells, such as HEK293F cells growing in suspension under serum-free conditions, are transfected with one or more vector(s) adapted for the production of a lentiviral particle. Preferably, the transfection is a transient transfection.

The different functions necessary for the production of a lentiviral particle can be provided to the packaging cells by any number of vectors. In particular, these functions may be provided by at least one, two, three or four vectors. In a particular embodiment of the invention, the different functions necessary for production of a lentiviral particle are provided to the packaging cell by the transfection, in particular transient transfection, of four vectors adapted for producing lentiviral particles, wherein one vector encodes envelope proteins (Env vector), one vector encodes lentiviral Gag and Pol proteins (Gag-Pol vector), one vector encodes a lentiviral Rev protein (Rev vector) and one vector is the closed linear transfer vector of the present invention comprising a transgene expression cassette between sequences encoding the lentiviral 5′ hybrid LTR and 3′ SIN LTR.

Alternatively, the closed linear transfer vector of the present vector may be transiently transfected into a stable producer cell bearing all or part of the complementary set of packaging functions required to produce an infectious lentiviral particle.

Various techniques known in the art may be employed for introducing nucleic acid molecules into packaging or producer cells. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”).

However, according to a preferred embodiment of the invention, transient transfection is carried out using polyethylenemine (PEI) as a transfection reagent. PEI is a synthetic, water-soluble polymer and is widely used as a transfection reagent. PEI has high gene transfer activity in many cell lines while displaying low cytotoxicity, is cost-effective and therefore is compatible with industrial scale production applications. PEI is available as both a linear and branched polymer with a wide range of molecular weights and polydispersities, physicochemical parameters that are critical for efficient gene transfer activity (Godbey W. T. et al., J. Control Release, 60, 149160 (1999)). In a particular embodiment, the PEI used in the present invention is a 20-25 kD linear PEI. For example, in a particular embodiment, the PEI used in the present invention is PEIPro® (available from PolyPlus). PEIPro® transfection reagents are linear PEI derivatives, free of components of animal origin, providing highly effective and reproducible gene delivery. Other PEI or cationic polymers similar in structure thereto for transfecting cells are disclosed in U.S. Pat. No. 6,013,240 and EP Patent No. 0770140.

The person skilled in the art can adapt the transfection method to the particular cell culture implemented.

Packaging cells may be transfected with the closed linear transfer vector of the present invention, along with one or more production vectors. The production vectors may be in any appropriate form, including closed linear DNA (with or without a 3′ spacer sequence as described herein), or circular DNA, such as a plasmid or minicircle. The production vectors may encode one or more of the packaging elements GAG, POL, REV and/or ENV. It may be preferable for GAG and POL to be encoded on a single production vector.

A packaging cell may be transfected with the closed linear transfer vector and the one or more production vectors using any appropriate molar construct ratio. For example, wherein a packaging cell is transfected with 4 DNA constructs (the closed linear transfer vector, GagPol vector, Rev vector and ENV vector (preferably VSVg vector)), any appropriate construct mass ratio may be used. The construct mass ratio of transfer:GagPol:Rev:VSVg DNA constructs may be 4:1:2:1, 3:1:2:1, 3:1:3:1.5, or 3:1:3:2.

### Induction

In embodiments of the present invention wherein an inducible system is used for the production of lentiviral particles, the packaging cell or producer cell containing the closed linear transfer vector of the present invention may be induced to begin production of the lentiviral particle. Inducible systems are well known in the art, for example Tet-on and Tet-off systems, which are based on the addition or removal, respectively of the tetracycline/doxycycline antibiotic in the culture medium to trigger gene transcription through the tetracycline response element (TRE). Alternative inducible systems include, but are not limited to, Tet-on/cumate inducible system and ecdysone inducible system,

In alternative embodiments of the present invention, a constitutive system may be used for the production of lentiviral particles.

### Culturing

After transfection, for example after adding the mixture of DNA and PEI to the cell culture, this cell culture is allowed to grow for a time which can be comprised between 36 and 72 hours post-transfection, in particular after 48 hours.

Methods for culturing the transfected packaging cell or producer cell are known in the art and include the use of various cell culture media, appropriate gas concentration/exchange and temperature control to promote growth of the cells and integration of the constructs into the genome of the cell.

In a particular embodiment, the medium used for culturing the packaging cells or producer cells is the same as the medium used for transfecting said cells. For example, in case of a transfection with a mixture of PEI and vector(s), the mixture may be done in Gibco LV-MAX Production Media (ThermoFisher Scientific, catalogue No. A3583401) and the cells may also be grown in said Gibco LV-MAX Production Media (ThermoFisher Scientific, catalogue No. A3583401) after transfection.

Culture may be carried out in a number of culture devices such as bioreactors adapted to the culture of cells in suspension. The bioreactor may be a single-use (disposable) or reusable bioreactor. The bioreactor may for example be selected from culture vessels or bags and tank reactors. Non-limiting representative bioreactors include Ambr15 (Sartorius), Ambr250 (Sartorius) iCELLis fixed bed bioreactor (Pall Life Sciences), Scale-X hydro (Univercells), HyPerforma Single-Use Bioreactor (ThermoScientific).

### Harvesting

The lentiviral particle may then be harvested (or collected), with one or more harvesting step using standard techniques well known in the art.

The total particle, infectious, and genomic titres can be determined by standard methods known in the art, including, but not limited to those demonstrated in the Examples below.

Thus, the invention provides a novel closed linear DNA vector suitable for production of lentiviral particles. The invention furthermore relates to a method of generating infectious lentiviral particles using the construct.

The invention will now be described with reference to the following non-limiting examples.

### EXAMPLES

### Materials and MethodsPlasmid/Closed Linear DNA Cloning and Manufacture

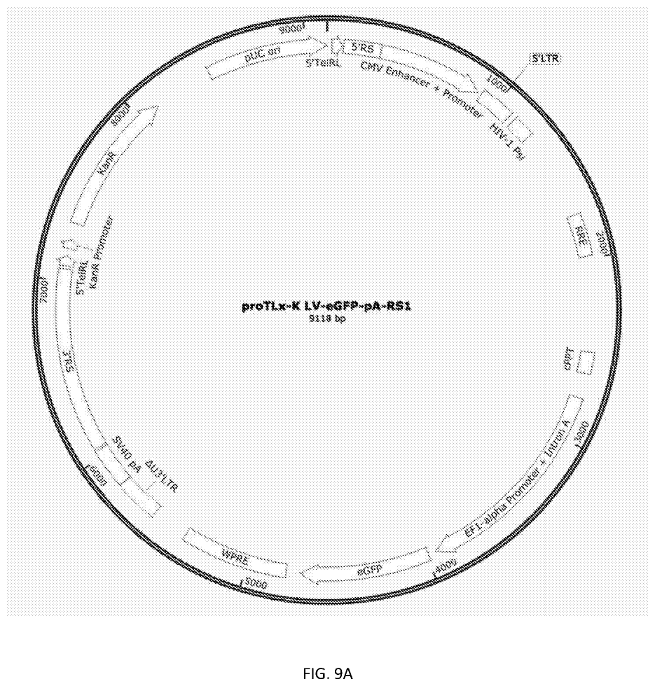

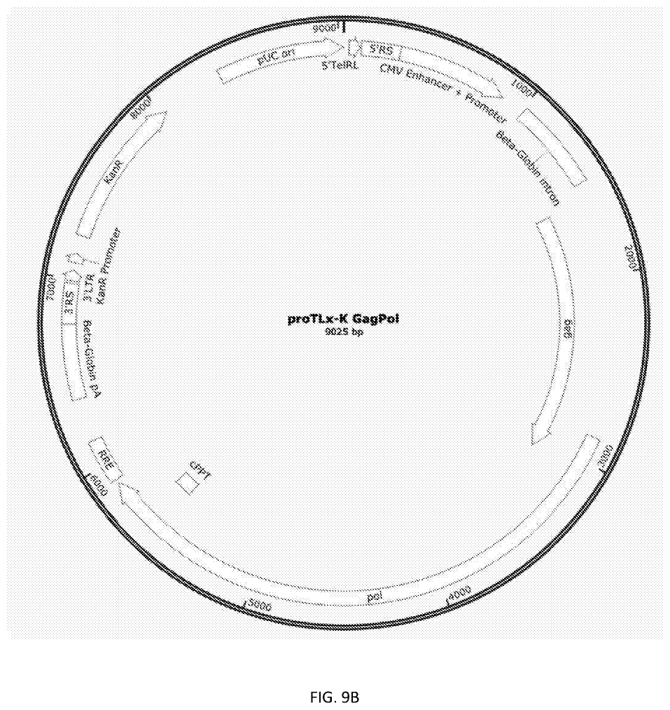

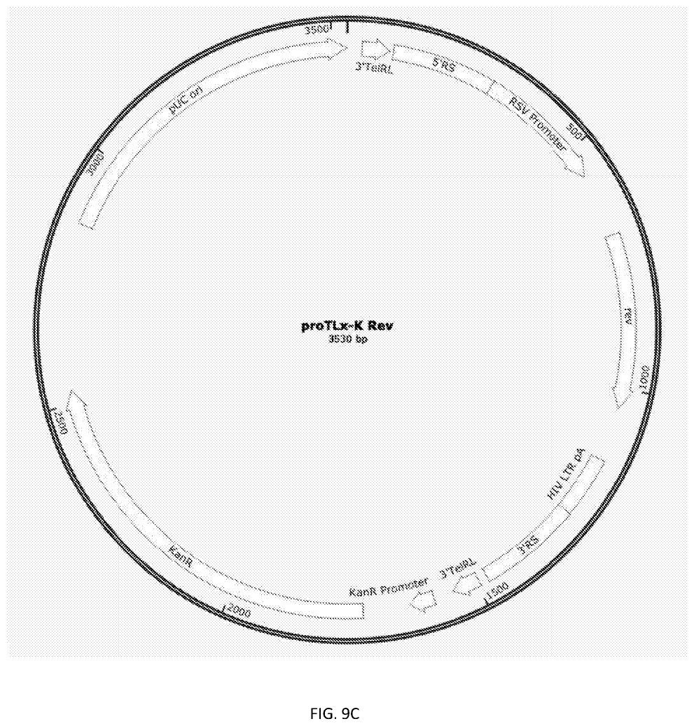

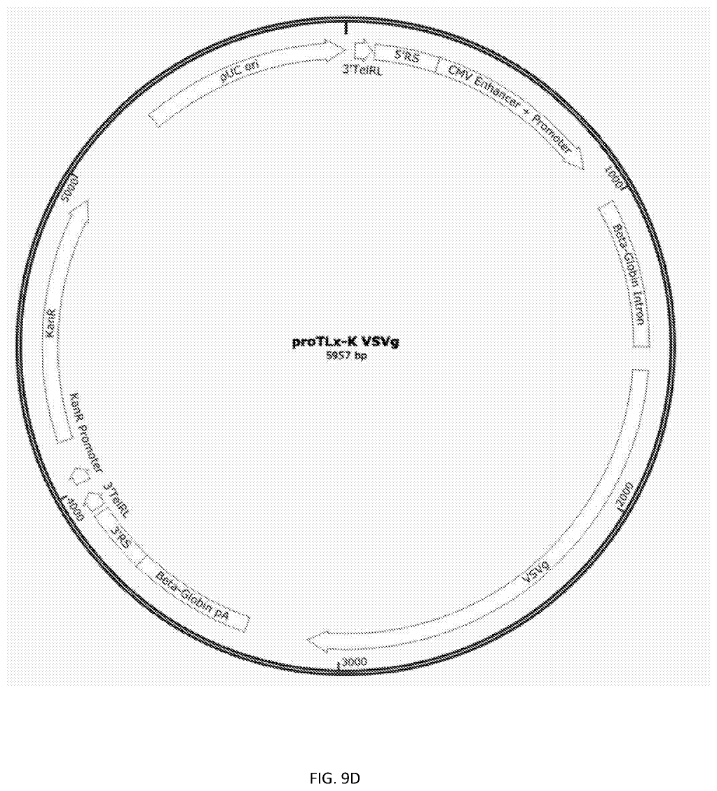

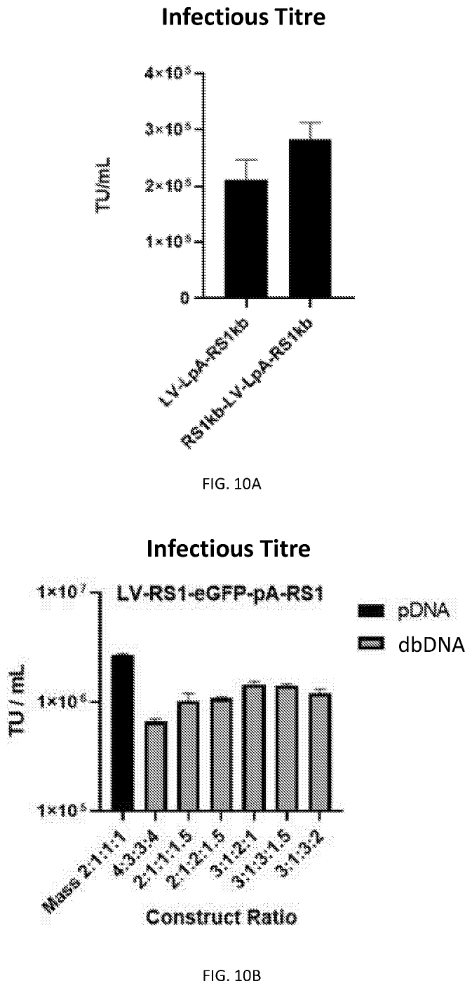

All sequences for the standard DNA lentiviral constructs (eGFP transgene, GagPol, Rev and VSG) were obtained from a publicly available source, Addgene (www.addgene.org) choosing from widely used lentiviral 3rd generation production systems. Those sequences were synthesized de novo and cloned into Touchlight's proTLx backbone (FIGS. 9A to D). The resulting plasmids (pDNA) were used as templates to generate the equivalent closed linear DNA versions through Touchlight's dbDNA manufacturing process (WO2010/086626). The various closed linear DNA constructs made are depicted in FIG. 4.

All modifications to the standard eGFP transgene to include the new elements described here were also synthesized de novo and underwent the same procedure for pDNA and closed linear DNA manufacturing at Touchlight (Hampton, UK).

The CAR-T gene was designed based on the 1928z sequence described by the Sadelain lab (Eyquem, J. et al. Targeting a CAR to the TRAC locus with CRISPR/Cas9 enhances tumour rejection. Nature 543, (2017)).

### Lentiviral Production: Cell Culture, Transfection and Harvest

For all lentiviral productions, HEK293F cells (Gibco Viral Production Cells, A35347) were cultured following manufacturer's recommendations in Erlenmeyer flasks with vent cap using LV-MAX Production Media (A3583401) at 50-100 mL volumes in a platform shaking incubator at 37° C., 8% CO₂ and 125 rpm.

The day before transfection, 50 mL cultures were established at a concentration of 1×10⁶ cells/mL. On the day of transfection, a total of 0.5-1 g/mL DNA containing the 4 lentiviral production constructs—eGFP transfer vector, GagPol, Rev, and VSVg—was transfected using PEIPro (PolyPlus Transfection) as transfection reagent following manufacturer's recommendations for suspension cells.

Harvest was performed 48h/72h post-transfection by filtering the supernatants (0.45 m) after centrifugation of the 50 mL cultures for 5 min at 1300 rpm. Supernatants were subsequently aliquoted and stored at −80° C. for later analysis. Cell pellets were resuspended and washed with 50 mL PBS (Sigma Aldrich, D8537) that then used for cell density (Trypan Blue), analysis of cellular eGFP expression using CytoFlex Flow Cytometer (Beckman Coulter) and cell pellet −80° C. storage for later gene expression analysis.

### DNA Delivery and Gene Expression Analysis

From the packaging cell pellets, total DNA and RNA was extracting using DNeasy Blood and Tissue and RNeasy Plus Mini kits respectively from Qiagen (www.qiagen.com) following the recommended protocols. For DNA delivery, extracted total DNA was then analysed by singleplex qPCR analysis with a StepOnePlus qPCR (Applied Biosystems) using, in separate reactions, a custom TaqMan primers/probe set (IDT Technologies) against the lentiviral target sequence, together with a copy number standard curve using the adequate reference material, and the RNAseP TaqMan Copy Number Reference Assay (Applied Biosystems) together with a wild type HEK293F genomic DNA standard curve to assess the number of DNA vector copies delivered per cell during transfection. For gene expression analysis, lug RNA was used to synthesize cDNA with SuperScript Ill First-Strand Synthesis SuperMix for qRT-PCR (Thermo Fisher Scientific). cDNA was then analysed by duplex qPCR analysis using a custom FAM-dye TaqMan primers/probe set (IDT Technologies, https://eu.idtdna.com) against the lentiviral target sequence and a gene expression housekeeping gene, either GAPDH/18S VIC-dye endogenous control (Applied Biosystems) together with a copy number standard curve using the adequate reference material to assess the normalized number of transcripts being generated. Prior to these assays, several TaqMan Primers/Probe sets per target were designed using IDT's PrimerQuest online tool (www.idtdna/primerquest) and then tested to select the best performing ones which sequences are described in the table below.. For eGFP, we used a validated TaqMan gene expression assay (FAM) from Applied Biosystems (4331182, Assay ID Mr04097229_mr).

TABLE 2
TagMan primer/probe sets from IDT Technologies

| Probe/ | | | 3' |
|---|---|---|---|
| Primer Name | 5' Dye | Sequence | Quencher |
| GAG1 FAM-BHQ1 | /56- | CTGGCCTGTTAGAAACATCAGAAGGC | /3BHQ_1/ |
| | FAM/ | T (SEQ ID NO. 1) | |
| GAG1 FW | None | GAGCTAGAACGATTCGCAGTTA (SEQ | None |
| | | ID NO. 2) | |
| GAG1 RV | None | CTGTCTGAAGGGATGGTTGTAG (SEQ | None |
| | | ID NO. 3) | |
| POL1 FAM-BHQ1 | /56- | TGGTCAGTGCTGGAATCAGGAAAGT | /3BHQ_1/ |
| | FAM/ | (SEQ ID NO. 4) | |
| POL1 FW | None | GTACCAGCACACAAAGGAATTG (SEQ | None |
| | | ID NO. 5) | |
| POL1 RV | None | ATGTTCTTCTTGGGCCTTATCT | None |
| | | (SEQ ID NO. 6) | |
| Rev1 FAM-BHQ1 | /56- | TATCAAAGCAACCCACCTCCCAATCC | /3BHQ_1/ |
| | FAM/ | (SEQ ID NO. 7) | |
| Rev1 FW | None | CAAGGCAGTCAGACTCATCAA (SEQ | None |
| | | ID NO. 8) | |
| Rev1 RV | None | TCTCTCTCCACCTTCTTCTTCT | None |
| | | (SEQ ID NO. 9) | |
| VSVg1 FAM-BHQ1 | /56- | ATTTCCGCTGGTATGGACCGAAGT | /3BHQ_1/ |
| | FAM/ | (SEQ ID NO. 10) | |
| VSVg1 FW | None | CCCAAGAGTCACAAGGCTATTC (SEQ | None |
| | | ID NO. 11) | |
| VSVg1 RV | None | GAGTGAAGGATCGGATGGAATG | None |
| | | (SEQ ID NO. 12) | |
| LTR-P FAM-BHQ1 | /56- | CAGTGGCGCCCGAACAGGGA (SEQ ID | /3BHQ_1/ |
| | FAM/ | NO. 13) | |
| LTR MH531 FW | None | TGTGTGCCCGTCTGTTGTGT (SEQ ID | None |
| | | NO. 14) | |
| LTR MH532 RV | None | GAGTCCTGCGTCGAGAGAGC (SEQ ID | None |
| | | NO. 15) | |

### Lentiviral Samples Analysis: Total Titre, Infectious Titre and Genomic Titre

For assessing total titres (Lentiviral particles per mL, LP/mL) from diluted lentiviral supernatants, a Lentivirus-Associated p24 ELISA Kit (Cell Biolabs, VPK-107-5) was used following the instructions provided by the manufacturer.

For measuring the infectious titre (Transduction Units per mL, TU/mL), adherent HEK293T (Lenti-XTM 293T, Takara, 632180) were cultured and seeded the day before in 6-well plates and, on infection day, exposed to different dilutions of the lentiviral supernatants with 12 μg/mL of Polybrene (Santa Cruz, sc-134220). Plates were centrifuged at 900×g for 30 min at room temperature and then incubated for 72 h at 37° C. and 5% CO₂. 72h post-infection, cells were trypsinised, washed with PBS and cellular eGFP expression was analysed by Cytoflex Flow Cytometer (Beckman Coulter). Supernatant dilutions giving 5-25% of eGFP positive cells were used to calculate infectious titre (TU/mL) using the following formula: TU/mL=(F×C/V)×D, where F=frequency of GFP+ cells (% GFP+ cells/100), C=cell number per well seeded for transduction V=volume of inoculum in mL (0.1 mL) and D=lentivirus dilution factor.

For measuring infectious titre of CAR19hCD28z LVV, 5×105 THP-1 cells were seeded per well of a 24-well plate on the day of infection. Cells were infected with serial dilutions of LVV supernatants in medium containing 8ug/mL polybrene and centrifuged at 1000×g for 1 h at RT. 48 h after infection, cells were washed and stained with anti-mouse F(ab′)2 fragment IgG conjugated with Alexa Fluor 647 and analysed by FACS, as above, to determine CAR19h28z expression. Infectious titre was calculated as described above.

Genomic titre (Genome particles per mL, GP/mL) was calculated using Takara's Lenti-X qRT-PCR Titration Kit (631235) that requires genome RNA extraction for the lentiviral supernatants and posterior lentiviral genome copies quantification by qRT-PCR.

Gene Expression Analysis by qRT-PCR

Total RNA was extracted from the cell pellets collected during lentiviral harvest using RNeasy Plus Mini kit (Qiagen, 74134) following manufacturer's protocol for animal cells. From 1 g of total RNA, cDNA synthesis was carried out using SuperScript Ill First-Strand Synthesis SuperMix for qRT-PCR (ThermoFisher Scientific, 11752050). A copy number standard curve (from 10⁸ to 10² copies/well), made from dbDNA of the transgene construct, was run in a StepOnePlus Real-Time PCR System (Applied Biosystems, 4376600) side by side with diluted cDNA from harvest samples. The qPCR runs were performed using Fast Advanced Master Mix (ThermoFisher Scientific, 4444556) by duplexing with a FAM dye primers/probe set for full length genomic RNA (LTR-P set: oligos MH531-5′TGTGTGCCCGTCTGTTGTGT 3′ (SEQ ID NO. 14) and MH532-5′ GAGTCCTGCGTCGAGAGAGC 3′ (SEQ ID NO. 15), and fluorescent probe LRT-P (5′ FAM-CAGTGGCGCCCGAACAGGGA-BHQ 3′ (SEQ ID NO. 13); Integrated DNA Technologies) or total RNA from transgene (Enhanced GFP, FAM TaqMan Gene expression assay; Applied Biosystems, 4351370, Assay ID Mr04097229_mr) and a VIC dye primers/probe set against Eukaryotic 18S rRNA Endogenous Control (VIC/MGB probe; Applied Biosystems, 4319413E). The endogenous control was used for sample normalisation and transcript copy number was calculated for each sample from the copy number standard curve.

### Example 1

### Results

### Prior Art Constructs

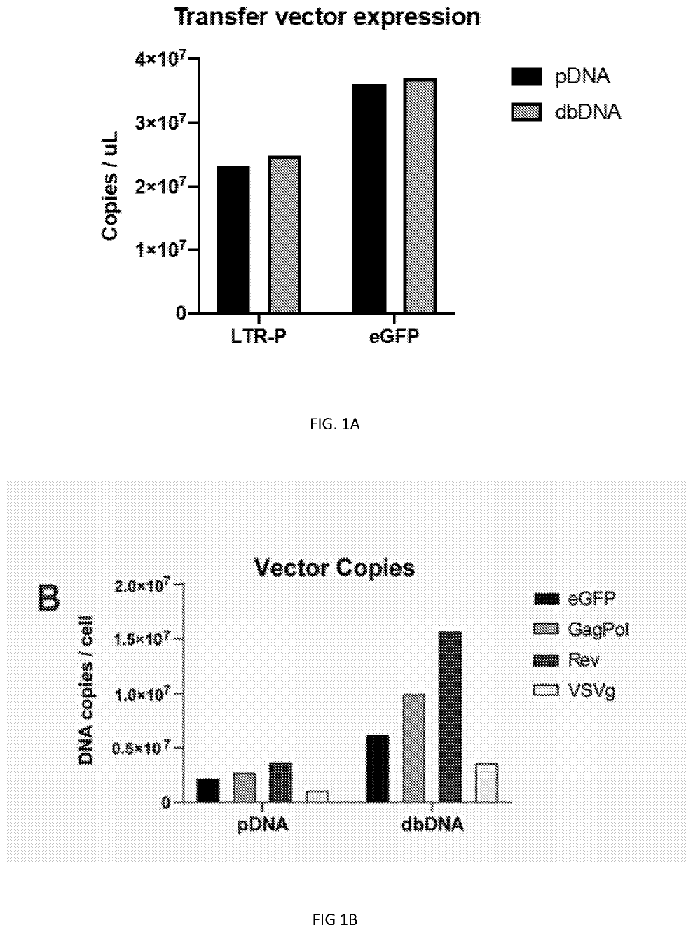

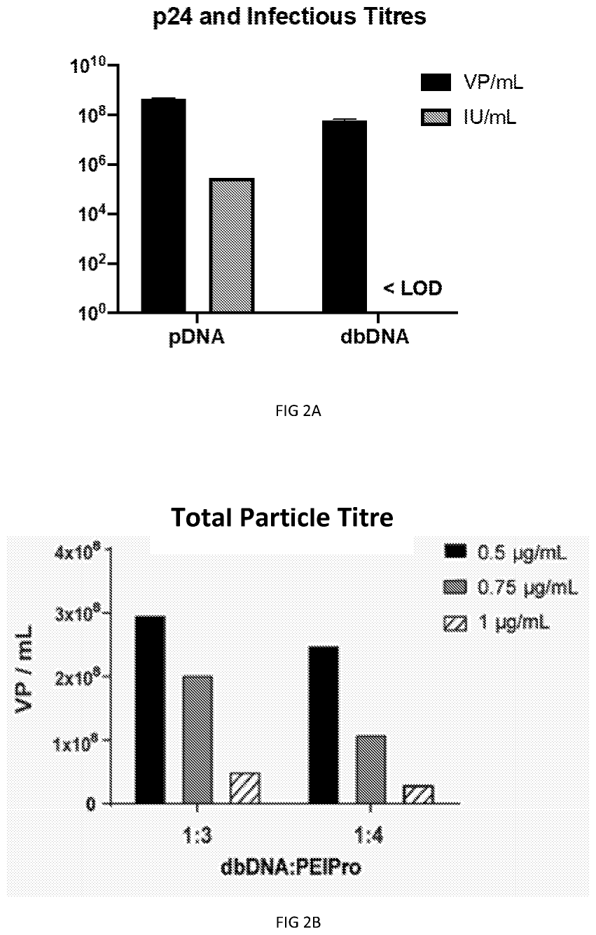

FIG. 1A depicts gene expression in producer cells 72h post transfection with a standard EF1α-eGFP-WPRE transfer vector, and lentiviral packaging constructs. RNA extracted from cells was subjected to RT-qPCR using the probes LTR-P and eGFP to quantify full length genomic RNA transcripts and total RNA transcripts, respectively. Transfer vector gene expression from closed linear DNA (dbDNA™) constructs was similar as for corresponding plasmid DNA (pDNA), demonstrating that low infectious titres were not the result of insufficient transfer vector RNA. The unique structure of the closed linear DNA vector is thought to alter transfection and expression in producer cells, negatively affecting titres. RT-qPCR was used to analyse DNA copy number as well as transcript abundance in producer cells 72 hours post transfection. This revealed that cells transfected with closed linear DNA contained 3-4-fold more DNA copies per cell of each construct compared to plasmid (FIG. 1B) FIG. 2 depicts the titres from production using “standard” transfer vectors, without a poly(A) sequence and a spacer. Total viral titre (p24) was five times lower for closed linear DNA (dbDNA) transfection than for the corresponding plasmid DNA (pDNA) transfection. However, the infectious viral titre for the closed linear DNA (dbDNA) transfection was below the limit of detection, demonstrating that in this instance very few infectious viral particles are produced. Key: VP/ml is viral particle per millilitre and TU/ml is Transducing Units per millilitre (infectious titre).

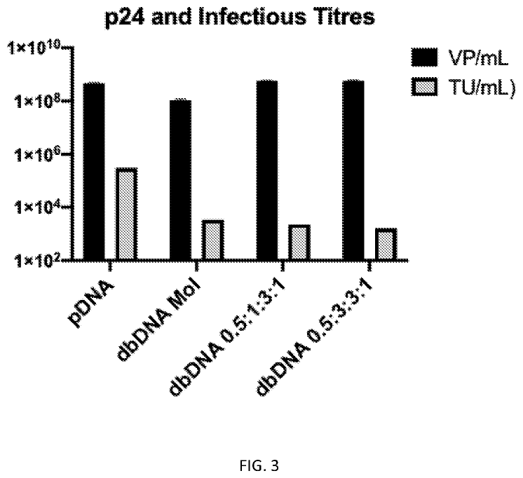

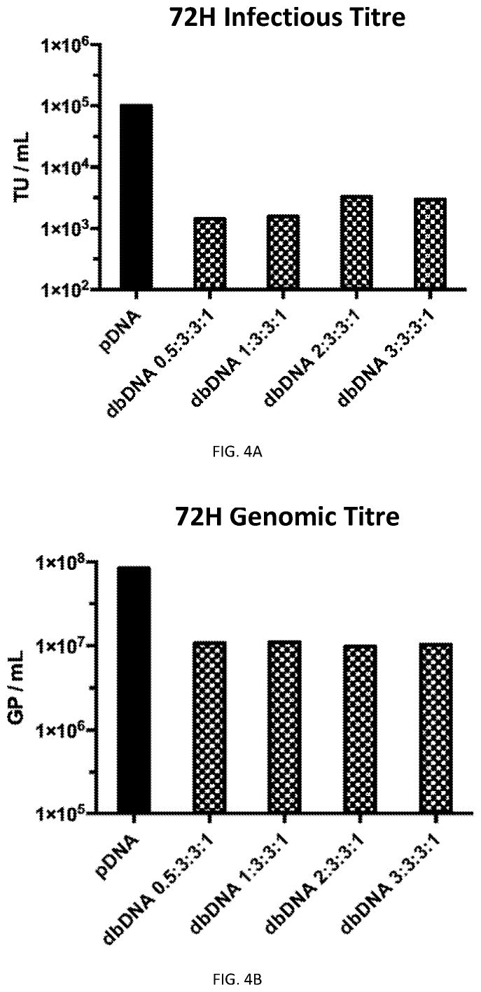

Following these results, attempts were made to optimise the conditions for closed linear DNA transfection, and the results from these experiments can be seen in FIG. 3. Optimising conditions for closed linear DNA transfection resulted in a complete rescue of total particle titres (p24), but infectious titres remained 100 times lower for closed linear DNA than plasmid.

Further optimisation work was conducted to see if increasing the amount of the transgene payload would increase infectious titre. Results shown in FIGS. 4A and 4B indicate that increasing transgene does not rescue the infectious titre. The results from the genomic titre assay (FIG. 4B) suggest that packaging of viral genomes into particles is inefficient for closed linear DNA compared to pDNA, and increasing the amount of transfer vector does not improve this situation.

### Novel Architectures

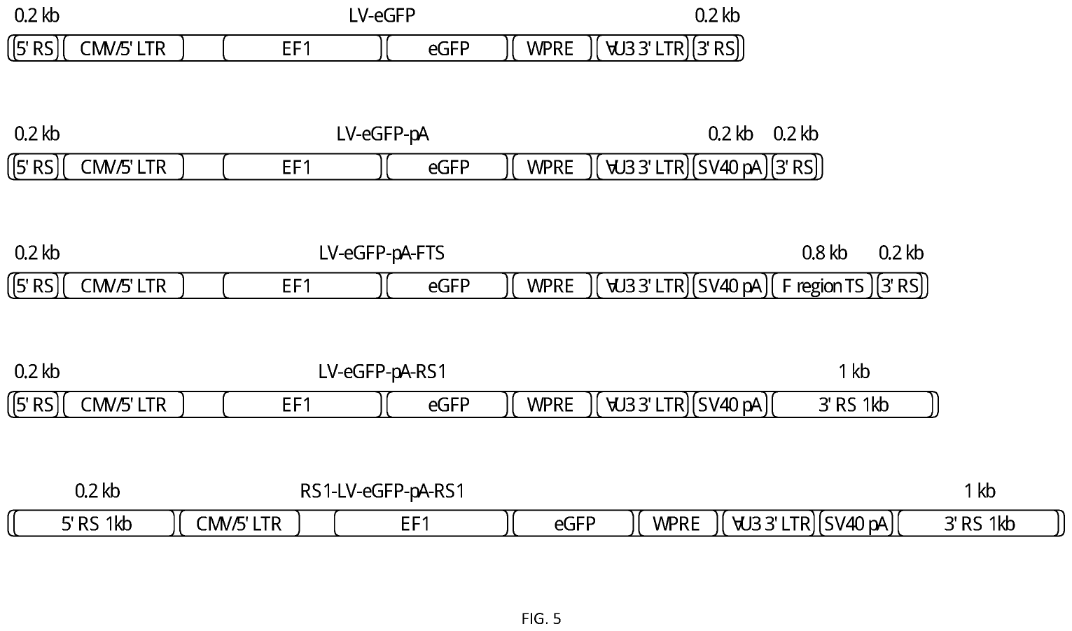

The novel closed linear DNA architectures depicted in FIG. 5 were constructed (as above) and tested in transfection experiments (as above). FIG. 6 (A-C) shows the effect on the total particle titre, infectious titre, and genomic titre of the different constructs. Cleaving downstream of 3′ LTR using Avrll restriction digest does not improve titres, suggesting that there is no read-through interference. However, addition of SV40 p(A) improves both pDNA and closed linear DNA lentiviral vector titres.

FIG. 7 (A-C) show the results where adding a spacer to the closed linear DNA construct (either the F region termination sequence (FTS) or the 1kb spacer (RS1)) further improves both infectious and genomic titres from SV40 poly(A) alone. As it can be seen, this effect is only seen in closed linear DNA vector and not plasmid. It is also clear that the effect is not dependent on sequence of the spacer.

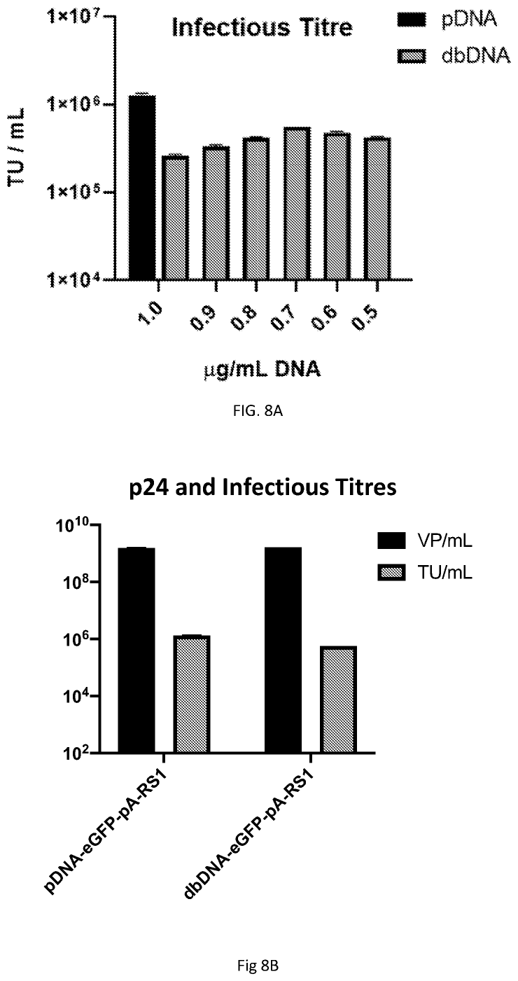

FIG. 8 shows a further optimisation of routine transfection conditions for closed linear DNA vectors in relation to infectious titre when compared to pDNA, which resulted in an infectious titre only two-fold lower than plasmid.

### Example 2

### 5′Spacer Sequence

The novel closed linear DNA architectures were constructed (as above) and tested in transfection experiments (as above). FIG. 10A show the results where adding a 1kb random spacer sequence (RS1) upstream of the CMV/5′ LTR in addition to the 3′ SV40 poly(A) and 3′ spacer (LV-RS1-eGFP-pA-RS1 further improves both infectious and genomic titres from SV40 poly(A) and 3′ spacer sequence. Addition of the 5′ spacer sequence led to a further 2-fold improvement in infectious titre.

### Optimised Closed Linear Production Vector

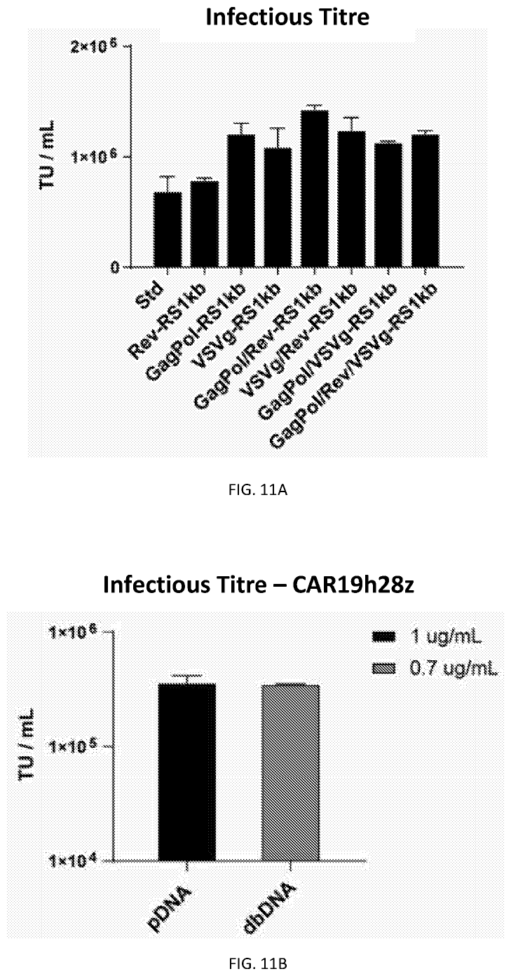

The closed linear production vectors were constructed (as above) and tested in transfection experiments (as above). Productions were carried out in which each production construct was swapped for the equivalent accessory+3′ RS1kb, individually or in groups. FIG. 11A shows the results where including a 3′ spacer sequence in a closed linear production vector improves infectious titres. Addition of RS1kb to either GagPol or VSVg substantially improved infectivity. The combination of GagPol-RS1kb and Rev-RS1kb yielded the highest titres.

### Example 3—Construct Ratio Optimisation

High-throughput optimisation of construct ratios for the 4 DNA constructs an eGFP payload, GagPol, Rev, and VSVg in the context of the novel transfer vector architecture (LV-RS1-eGFP-pA-RS1) was performed using 0.7 g/mL total input transfer vector. Several conditions in which transfer vector and Rev were increased yielded significant improvements in infectious titres over our previous condition of 4:3:3:4. In particular, titres of up to 1.4×10⁶ TU/mL were achieved using the ratio of 3:1:2:1 (FIG. 10B)

### Example 4—CAR19h28z Lentiviral Particle

A lentiviral particle expressing CAR19h28z, including the downstream SV40 LpA and flanking RS1kb (LV-RS1kb-1928z-LpA-RS1kb) was generated. HEK293F suspension cells were co-transfected with LV-RS1kb-1928z-LpA-RS1kb, GagPol-RS1kb, Rev-RS1kb, and VSVg at a molar ratio of 4:1:2:1 for dbDNA (0.7 ug/mL DNA; 1:3 DNA:PEI), and a mass ratio of 2:1:1:1 for plasmid (1 ug/mL DNA; 1:2 DNA:PEI) and supernatants were harvested 72h later for infectious titre analysis by CD19 FACS of transduced THP1 cells. As shown in FIG. 10B, using the fully optimised suite of constructs and optimised transfection condition, infectious titres for LVP CAR1928z were equivalent whether using plasmid or dbDNA as starting material. Taken together these data demonstrate that closed linear DNA can be used as an alternative starting material to plasmid for the manufacture of high titre LV.

### Example 5

### Optimising Total Vector Input and Construct Ratios Rescues Total Particle Titres.

This example used standard prior art closed linear DNA constructs. However, the beneficial effect is also demonstrated with the improved constructs (Example 3 and 4).

The observed difference in closed linear DNA expression profile indicated that transfection conditions and construct ratios needed to be further optimised to achieve titres equivalent to industry standard (plasmid). Using the construct ratio 2:1:1:1, total DNA input was assessed by transfecting cells with 0.5, 0.75, or 1.0 μg/mL closed linear DNA vectors. Samples were harvested at 72h post transfection for analysis of transfection efficiency and total p24 titre. A clear, dose-dependent increase in total particle titre was observed as total DNA was reduced (FIG. 2B, demonstrating a 6-fold increase in total particle titre using the lowest total input DNA).

Low Infectious Particle Titres are not Improved with Increased Transfer Vector.

Having identified conditions that yielded high particle titres, the infectivity of closed linear-derived particles was compared to plasmid. A comparability study was performed between the standard plasmid condition (2:1:1:1; 1 μg/mL), and closed linear vectors (0.5 μg/mL) using the molar equivalent to plasmid, and the 2 optimised ratio conditions of 0.5:1:3:1 and 0.5:3:3:1. This confirmed that total particle titres using the optimised closed linear ratio conditions were reproducibly equivalent to plasmid, however infectious titres were found to be approximately 100-fold lower (FIG. 3). Low infectious titres correlated with a low abundance of lentiviral genomic RNA (vgRNA), suggesting that particles may be empty due to insufficient transfer vector. This supports the hypothesis that the low infectivity of closed linear DNA-derived LV is related to low packaging efficiency.

1. A closed linear DNA vector suitable for use as a lentiviral transfer vector comprising sequences in the following order 5′ to 3′:
(a) a hybrid 5′ long terminal repeat (LTR) sequence;
(b) a promoter operably linked to a transgene;
(c) a 3′ self-inactivating (SIN) sequence;
(d) a poly(A) signal sequence; and
(e) a spacer sequence.

2. A closed linear DNA vector as claimed in claim 1 wherein said spacer sequence is at least 250 nucleotides in length.

3. A closed linear DNA vector as claimed in claim 1, wherein the promoter and transgene are flanked by the 5′ and 3′ LTR sequences.

4. A closed linear DNA vector as claimed in claim 1, wherein said closed linear DNA vector further comprises one or more further spacer sequences, preferably a 5′ spacer sequence located 5′ to the hybrid 5′ LTR sequence.

5. A closed linear DNA vector as claimed in claim 4 wherein said 5′ spacer sequence is at least 250 nucleotides in length.

6. A closed linear DNA vector as claimed in claim 1 wherein said hybrid 5′LTR has all or part of the U3 region replaced with a heterologous promoter.

7. A closed linear DNA vector as claimed in claim 1, wherein said poly(A) signal sequence includes additional helper sequences, optionally wherein said helper sequences are one or more upstream sequence elements (USE).

8. A closed linear DNA vector as claimed in claim 1 wherein said poly(A) signal is a strong poly(A) signal.

9. A closed linear DNA vector as claimed in claim 1 wherein said poly(A) signal is selected from the SV40 Late poly(A) sequence, rabbit β-globin poly(A) (rbGlob), or bovine growth hormone poly(A) (bGHpA), or a sequence having at least 90% homology to said sequences.

10. A closed linear DNA vector as claimed in claim 1, wherein said 3′ SIN LTR contain one or more deletions compared to a wild-type LTR, optionally a deletion in the U3 region.

11. A closed linear DNA vector as claimed in claim 10, wherein said 3′ SIN LTR contains a 133 nucleotide U3 deletion compared to a wild-type 3′ LTR nucleotides −149 to −9 with respect to transcription start site.

12. A method for improving the infectious titre of lentiviral particles when the transfer vector is a closed linear DNA vector, comprising introducing a closed linear DNA vector as claimed in claim 1 to a packaging cell or producer cell.

13. The method according to claim 12, further comprising introducing to the packaging cell one or more production vectors encoding viral elements required for the manufacture of lentiviral particles.

14. The method according to claim 13, wherein the one or more production vectors include one or more of the following:
(e) a lentiviral group specific antigen (GAG) gene; and/or
(f) a lentiviral polymerase (POL) gene; and/or
(g) an envelope gene (ENV); and/or
(h) a lentiviral regulatory gene (REV).

15. The method according to claim 14 wherein one or more of said production vectors are closed linear DNA vectors which optionally include a spacer sequence.

16. The method according to claim 15, wherein said closed linear DNA vector comprises:
(a) at least one expression cassette including one or more of:
i. a lentiviral group specific antigen (GAG) gene;
ii. a lentiviral polymerase (POL) gene;
iii. an envelope gene (ENV); and/or
iv. a lentiviral regulatory gene (REV), and
(b) a spacer sequence located 3′ to the expression cassette.

17. The method according to claim 14, wherein the envelope gene is a Vesicular Stomatitis Virus Glycoprotein (VSV-G) gene.

18. The method according to claim 14, wherein the GAG gene and POL gene are included on a single production vector.

19. The method according to claim 12, wherein the packaging cell is HEK293 cells or a variant or derivative thereof.

20-21. (canceled)

22. The method according to claim 15, wherein the closed linear transfer vector, GAG-POL-encoding production vector, REV-encoding production vector and ENV-encoding production vector are supplied to the packaging cell at a construct molar ratio of 4:1:2:1.

23. The method according to claim 12, further comprising:
(a) inducing production of the lentiviral particles in packaging cells or producer cells transfected with at least one closed linear DNA vector adapted for the production of a lentiviral particle; and/or
(b) culturing the transfected packaging cells or producer cells; and
(c) harvesting/isolating the produced recombinant lentiviral particles from the culture medium.
